SEQUENCE LISTING (120 sequences)

SEQ ID NO: 1 - protein, 42 aa, Artificial Sequence
  Synthetic Peptide
HXEGTFTSDLSKQNLEEEEAVRLFIEWLKQGGPSSGAPPPSK

SEQ ID NO: 2 - protein, 40 aa, Artificial Sequence
  Synthetic Peptide
HGEGTFTSDLSKQMEEEAVRLFIEWLKNGGPSSGAPPPSK

SEQ ID NO: 3 - protein, 40 aa, Artificial Sequence
  Synthetic Peptide
HXEGTFTSDVSSYLEEEAAKEFIAWLVRGGPSSGAPPPSK

SEQ ID NO: 4 - protein, 40 aa, Artificial Sequence; identical to SEQ ID NO: 3
  Synthetic Peptide

SEQ ID NO: 5 - protein, 39 aa, Artificial Sequence
  Synthetic Peptide
HAEGTFTSDVSSYLEGQAAEFIVWLVKGRGKKGGPPPSK

SEQ ID NO: 6 - protein, 39 aa, Artificial Sequence
  Synthetic Peptide
HAEGTFTSDVSSYLEGQAAEFIVWLVKGRGKKGXPPPSK

SEQ ID NO: 7 - protein, 39 aa, Artificial Sequence
  Synthetic Peptide
HAEGTFTSDVSSYLEEQAAEFIVWLVKGRGKKGGPPPSK

SEQ ID NO: 8 - protein, 39 aa, Artificial Sequence
  Synthetic Peptide
HAEGTFTSDVSSYLEEQAAEFIVWLVKGRGKKGXPPPSK

SEQ ID NO: 9 - protein, 39 aa, Artificial Sequence
  Synthetic Peptide
HAEGTFTSDVSSYLEXQAAEFIVWLVKGRGKKGGPPPSK

SEQ ID NO: 10 - protein, 39 aa, Artificial Sequence
  Synthetic Peptide
HAEGTFTSDVSSYLEXQAAEFIVWLVKGRGKKGXPPPSK

SEQ ID NO: 11 - protein, 39 aa, Artificial Sequence
  Synthetic Peptide
HAEGTFTSDVSSYMEGQAAEFIVWLVKGRGKKGGPPPSK

SEQ ID NO: 12 - protein, 39 aa, Artificial Sequence
  Synthetic Peptide
HAEGTFTSDVSSYMEEQAAEFIVWLVKGRGKKGGPPPSK

SEQ ID NO: 13 - protein, 39 aa, Artificial Sequence
  Synthetic Peptide
HAEGTFTSDVSSYMEEQAAEFIVWLVKGRGKKGXPPPSK

SEQ ID NO: 14 - protein, 39 aa, Artificial Sequence
  Synthetic Peptide
HAEGTFTSDVSSYMEXQAAEFIVWLVKGRGKKGGPPPSK

SEQ ID NO: 15 - protein, 39 aa, Artificial Sequence
  Synthetic Peptide
HAEGTFTSDVSSYMEXQAAEFIVWLVKGRGKKGXPPPSK

SEQ ID NO: 16 - protein, 39 aa, Artificial Sequence
  Synthetic Peptide
HAEGTFTSDVSSQLEGQAAEFIVWLVKGRGKKGGPPPSK

SEQ ID NO: 17 - protein, 39 aa, Artificial Sequence
  Synthetic Peptide
HAEGTFTSDVSSQLEGQAAEFIVWLVKGRGKKGXPPPSK

SEQ ID NO: 18 - protein, 39 aa, Artificial Sequence
  Synthetic Peptide
HAEGTFTSDVSSQLEEQAAEFIVWLVKGRGKKGGPPPSK

SEQ ID NO: 19 - protein, 39 aa, Artificial Sequence
  Synthetic Peptide
HAEGTFTSDVSSQLEEQAAEFIVWLVKGRGKKGXPPPSK

SEQ ID NO: 20 - protein, 39 aa, Artificial Sequence
  Synthetic Peptide
HAEGTFTSDVSSQLEXQAAEFIVWLVKGRGKKGGPPPSK

SEQ ID NO: 21 - protein, 39 aa, Artificial Sequence
  Synthetic Peptide
HAEGTFTSDVSSQLEXQAAEFIVWLVKGRGKKGXPPPSK

SEQ ID NO: 22 - protein, 39 aa, Artificial Sequence
  Synthetic Peptide
HAEGTFTSDVSSQMEGQAAEFIVWLVKGRGKKGGPPPSK

SEQ ID NO: 23 - protein, 39 aa, Artificial Sequence
  Synthetic Peptide
HAEGTFTSDVSSQMEGQAAEFIVWLVKGRGKKGXPPPSK

SEQ ID NO: 24 - protein, 39 aa, Artificial Sequence
  Synthetic Peptide
HAEGTFTSDVSSQMEEQAAEFIVWLVKGRGKKGGPPPSK

SEQ ID NO: 25 - protein, 39 aa, Artificial Sequence
  Synthetic Peptide
HAEGTFTSDVSSQMEEQAAEFIVWLVKGRGKKGXPPPSK

SEQ ID NO: 26 - protein, 39 aa, Artificial Sequence
  Synthetic Peptide
HAEGTFTSDVSSQMEXQAAEFIVWLVKGRGKKGGPPPSK

SEQ ID NO: 27 - protein, 39 aa, Artificial Sequence
  Synthetic Peptide
HAEGTFTSDVSSQMEXQAAEFIVWLVKGRGKKGXPPPSK

SEQ ID NO: 28 - protein, 39 aa, Artificial Sequence
  Synthetic Peptide
HAEGTFTSDVSKYLEGQAAEFIVWLVKGRGKKGGPPPSK

SEQ ID NO: 29 - protein, 39 aa, Artificial Sequence
  Synthetic Peptide
HAEGTFTSDVSKYLEGQAAEFIVWLVKGRGKKGXPPPSK

SEQ ID NO: 30 - protein, 39 aa, Artificial Sequence
  Synthetic Peptide
HAEGTFTSDVSKYLEEQAAEFIVWLVKGRGKKGGPPPSK

SEQ ID NO: 31 - protein, 39 aa, Artificial Sequence
  Synthetic Peptide
HAEGTFTSDVSKYLEEQAAEFIVWLVKGRGKKGXPPPSK

SEQ ID NO: 32 - protein, 39 aa, Artificial Sequence
  Synthetic Peptide
HAEGTFTSDVSKYLEXQAAEFIVWLVKGRGKKGGPPPSK

SEQ ID NO: 33 - protein, 39 aa, Artificial Sequence
  Synthetic Peptide
HAEGTFTSDVSKYLEXQAAEFIVWLVKGRGKKGXPPPSK

SEQ ID NO: 34 - protein, 39 aa, Artificial Sequence
  Synthetic Peptide
HAEGTFTSDVSKYMEGQAAEFIVWLVKGRGKKGGPPPSK

SEQ ID NO: 35 - protein, 39 aa, Artificial Sequence
  Synthetic Peptide
HAEGTFTSDVSKYMEGQAAEFIVWLVKGRGKKGXPPPSK

SEQ ID NO: 36 - protein, 39 aa, Artificial Sequence
  Synthetic Peptide
HAEGTFTSDVSKYMEEQAAEFIVWLVKGRGKKGGPPPSK

SEQ ID NO: 37 - protein, 39 aa, Artificial Sequence
  Synthetic Peptide
HAEGTFTSDVSKYMEEQAAEFIVWLVKGRGKKGXPPPSK

SEQ ID NO: 38 - protein, 39 aa, Artificial Sequence
  Synthetic Peptide
HAEGTFTSDVSKYMEXQAAEFIVWLVKGRGKKGGPPPSK

SEQ ID NO: 39 - protein, 39 aa, Artificial Sequence
  Synthetic Peptide
HAEGTFTSDVSKYMEXQAAEFIVWLVKGRGKKGXPPPSK

SEQ ID NO: 40 - protein, 39 aa, Artificial Sequence
  Synthetic Peptide
HAEGTFTSDVSKQLEGQAAEFIVWLVKGRGKKGGPPPSK

SEQ ID NO: 41 - protein, 39 aa, Artificial Sequence
  Synthetic Peptide
HAEGTFTSDVSKQLEGQAAEFIVWLVKGRGKKGXPPPSK

SEQ ID NO: 42 - protein, 39 aa, Artificial Sequence
  Synthetic Peptide
HAEGTFTSDVSKQLEEQAAEFIVWLVKGRGKKGGPPPSK

SEQ ID NO: 43 - protein, 39 aa, Artificial Sequence
  Synthetic Peptide
HAEGTFTSDVSKQLEEQAAEFIVWLVKGRGKKGXPPPSK

SEQ ID NO: 44 - protein, 39 aa, Artificial Sequence
  Synthetic Peptide
HAEGTFTSDVSKQLEXQAAEFIVWLVKGRGKKGGPPPSK

SEQ ID NO: 45 - protein, 39 aa, Artificial Sequence
  Synthetic Peptide
HAEGTFTSDVSKQLEXQAAEFIVWLVKGRGKKGXPPPSK

SEQ ID NO: 46 - protein, 39 aa, Artificial Sequence
  Synthetic Peptide
HAEGTFTSDVSKQMEGQAAEFIVWLVKGRGKKGGPPPSK

SEQ ID NO: 47 - protein, 39 aa, Artificial Sequence
  Synthetic Peptide
HAEGTFTSDVSKQMEGQAAEFIVWLVKGRGKKGXPPPSK

SEQ ID NO: 48 - protein, 39 aa, Artificial Sequence
  Synthetic Peptide
HAEGTFTSDVSKQMEEQAAEFIVWLVKGRGKKGGPPPSK

SEQ ID NO: 49 - protein, 39 aa, Artificial Sequence
  Synthetic Peptide
HAEGTFTSDVSKQMEEQAAEFIVWLVKGRGKKGXPPPSK

SEQ ID NO: 50 - protein, 39 aa, Artificial Sequence
  Synthetic Peptide
HAEGTFTSDVSKQMEXQAAEFIVWLVKGRGKKGGPPPSK

SEQ ID NO: 51 - protein, 39 aa, Artificial Sequence
  Synthetic Peptide
HAEGTFTSDVSKQMEXQAAEFIVWLVKGRGKKGXPPPSK

SEQ ID NO: 52 - protein, 39 aa, Artificial Sequence
  Synthetic Peptide
HAEGTFTSDLSKQLEGQAAEFIVWLVKGRGKKGGPPPSK

SEQ ID NO: 53 - protein, 39 aa, Artificial Sequence
  Synthetic Peptide
HAEGTFTSDLSKQLEGQAAEFIVWLVKGRGKKGXPPPSK

SEQ ID NO: 54 - protein, 39 aa, Artificial Sequence
  Synthetic Peptide
HAEGTFTSDLSKQLEEQAAEFIVWLVKGRGKKGGPPPSK

SEQ ID NO: 55 - protein, 39 aa, Artificial Sequence
  Synthetic Peptide
HAEGTFTSDLSKQLEEQAAEFIVWLVKGRGKKGXPPPSK

SEQ ID NO: 56 - protein, 39 aa, Artificial Sequence
  Synthetic Peptide
HAEGTFTSDLSKQLEXQAAEFIVWLVKGRGKKGGPPPSK

SEQ ID NO: 57 - protein, 39 aa, Artificial Sequence
  Synthetic Peptide
HAEGTFTSDLSKQLEXQAAEFIVWLVKGRGKKGXPPPSK

SEQ ID NO: 58 - protein, 39 aa, Artificial Sequence
  Synthetic Peptide
HAEGTFTSDLSKQMEGQAAEFIVWLVKGRGKKGGPPPSK

SEQ ID NO: 59 - protein, 39 aa, Artificial Sequence
  Synthetic Peptide
HAEGTFTSDLSKQMEGQAAEFIVWLVKGRGKKGXPPPSK

SEQ ID NO: 60 - protein, 39 aa, Artificial Sequence
  Synthetic Peptide
HAEGTFTSDLSKYLEGQAAEFIVWLVKGRGKKGGPPPSK

SEQ ID NO: 61 - protein, 39 aa, Artificial Sequence
  Synthetic Peptide
HAEGTFTSDLSKYLEGQAAEFIVWLVKGRGKKGXPPPSK

SEQ ID NO: 62 - protein, 39 aa, Artificial Sequence
  Synthetic Peptide
HAEGTFTSDLSKYMEGQAAEFIVWLVKGRGKKGGPPPSK

SEQ ID NO: 63 - protein, 39 aa, Artificial Sequence
  Synthetic Peptide
HAEGTFTSDLSKYMEGQAAEFIVWLVKGRGKKGXPPPSK

SEQ ID NO: 64 - protein, 39 aa, Artificial Sequence
  Synthetic Peptide
HAEGTFTSDLSSQLEGQAAEFIVWLVKGRGKKGGPPPSK

SEQ ID NO: 65 - protein, 39 aa, Artificial Sequence
  Synthetic Peptide
HAEGTFTSDLSSQLEGQAAEFIVWLVKGRGKKGXPPPSK

SEQ ID NO: 66 - protein, 39 aa, Artificial Sequence
  Synthetic Peptide
HAEGTFTSDLSSQMEGQAAEFIVWLVKGRGKKGGPPPSK

SEQ ID NO: 67 - protein, 39 aa, Artificial Sequence
  Synthetic Peptide
HAEGTFTSDLSSQMEGQAAEFIVWLVKGRGKKGXPPPSK

SEQ ID NO: 68 - protein, 39 aa, Artificial Sequence
  Synthetic Peptide
HAEGTFTSDLSSYLEGQAAEFIVWLVKGRGKKGGPPPSK

SEQ ID NO: 69 - protein, 39 aa, Artificial Sequence
  Synthetic Peptide
HAEGTFTSDLSSYLEGQAAEFIVWLVKGRGKKGXPPPSK

SEQ ID NO: 70 - protein, 39 aa, Artificial Sequence
  Synthetic Peptide
HAEGTFTSDLSSYMEGQAAEFIVWLVKGRGKKGGPPPSK

SEQ ID NO: 71 - protein, 39 aa, Artificial Sequence
  Synthetic Peptide
HAEGTFTSDLSSYMEGQAAEFIVWLVKGRGKKGXPPPSK

SEQ ID NO: 72 - protein, 39 aa, Artificial Sequence
  Synthetic Peptide
HKEGTFTSDVSSYLEGQAAEFIVWLVKGRGKKGGPPPSK

SEQ ID NO: 73 - protein, 39 aa, Artificial Sequence
  Synthetic Peptide
HKEGTFTSDVSSYLEGQAAEFIVWLVKGRGKKGXPPPSK

SEQ ID NO: 74 - protein, 39 aa, Artificial Sequence
  Synthetic Peptide
HKEGTFTSDVSSYLEEQAAEFIVWLVKGRGKKGGPPPSK

SEQ ID NO: 75 - protein, 39 aa, Artificial Sequence
  Synthetic Peptide
HLEGTFTSDVSSYLEEQAAEFIVWLVKGRGKKGXPPPSK

SEQ ID NO: 76 - protein, 39 aa, Artificial Sequence
  Synthetic Peptide
HLEGTFTSDVSSYLEXQAAEFIVWLVKGRGKKGGPPPSK

SEQ ID NO: 77 - protein, 39 aa, Artificial Sequence
  Synthetic Peptide
HKEGTFTSDVSSYLEXQAAEFIVWLVKGRGKKGXPPPSK

SEQ ID NO: 78 - protein, 39 aa, Artificial Sequence
  Synthetic Peptide
HKEGTFTSDVSSYMEGQAAEFIVWLVKGRGKKGGPPPSK

SEQ ID NO: 79 - protein, 39 aa, Artificial Sequence
  Synthetic Peptide
HIEGTFTSDVSSYMEGQAAEFIVWLVKGRGKKGXPPPSK

SEQ ID NO: 80 - protein, 39 aa, Artificial Sequence
  Synthetic Peptide
HIEGTFTSDVSSYMEEQAAEFIVWLVKGRGKKGGPPPSK

SEQ ID NO: 81 - protein, 39 aa, Artificial Sequence
  Synthetic Peptide
HIEGTFTSDVSSYMEEQAAEFIVWLVKGRGKKGXPPPSK

SEQ ID NO: 82 - protein, 39 aa, Artificial Sequence
  Synthetic Peptide
HLEGTFTSDVSSYMEXQAAEFIVWLVKGRGKKGGPPPSK

SEQ ID NO: 83 - protein, 39 aa, Artificial Sequence
  Synthetic Peptide
HXEGTFTSDLSSYMEGQAAEFIVWLVKGRGKKGXPPPSK

SEQ ID NO: 84 - protein, 39 aa, Artificial Sequence
  Synthetic Peptide
HKEGTFTSDVSSYMEXQAAEFIVWLVKGRGKKGXPPPSK

SEQ ID NO: 85 - protein, 39 aa, Artificial Sequence
  Synthetic Peptide
HIEGTFTSDVSSQLEGQAAEFIVWLVKGRGKKGGPPPSK

SEQ ID NO: 86 - protein, 39 aa, Artificial Sequence
  Synthetic Peptide
HVEGTFTSDVSSQLEGQAAEFIVWLVKGRGKKGXPPPSK

SEQ ID NO: 87 - protein, 39 aa, Artificial Sequence
  Synthetic Peptide
HVEGTFTSDVSSQLEEQAAEFIVWLVKGRGKKGGPPPSK

SEQ ID NO: 88 - protein, 39 aa, Artificial Sequence
  Synthetic Peptide
HVEGTFTSDVSSQLEEQAAEFIVWLVKGRGKKGXPPPSK

SEQ ID NO: 89 - protein, 39 aa, Artificial Sequence
  Synthetic Peptide
HLEGTFTSDVSSQLEXQAAEFIVWLVKGRGKKGGPPPSK

SEQ ID NO: 90 - protein, 39 aa, Artificial Sequence
  Synthetic Peptide
HKEGTFTSDVSSQLEXQAAEFIVWLVKGRGKKGXPPPSK

SEQ ID NO: 91 - protein, 39 aa, Artificial Sequence
  Synthetic Peptide
HKEGTFTSDVSSQMEGQAAEFIVWLVKGRGKKGGPPPSK

SEQ ID NO: 92 - protein, 39 aa, Artificial Sequence
  Synthetic Peptide
HVEGTFTSDVSSQMEGQAAEFIVWLVKGRGKKGXPPPSK

SEQ ID NO: 93 - protein, 39 aa, Artificial Sequence
  Synthetic Peptide
HVEGTFTSDVSSQMEEQAAEFIVWLVKGRGKKGGPPPSK

SEQ ID NO: 94 - protein, 39 aa, Artificial Sequence
  Synthetic Peptide
HGEGTFTSDVSSQMEEQAAEFIVWLVKGRGKKGXPPPSK

SEQ ID NO: 95 - protein, 39 aa, Artificial Sequence
  Synthetic Peptide
HGEGTFTSDVSSQMEXQAAEFIVWLVKGRGKKGGPPPSK

SEQ ID NO: 96 - protein, 39 aa, Artificial Sequence
  Synthetic Peptide
HIEGTFTSDVSSQMEXQAAEFIVWLVKGRGKKGXPPPSK

SEQ ID NO: 97 - protein, 39 aa, Artificial Sequence
  Synthetic Peptide
HIEGTFTSDVSKYLEGQAAEFIVWLVKGRGKKGGPPPSK

SEQ ID NO: 98 - protein, 39 aa, Artificial Sequence
  Synthetic Peptide
HKEGTFTSDVSKYLEGQAAEFIVWLVKGRGKKGXPPPSK

SEQ ID NO: 99 - protein, 39 aa, Artificial Sequence
  Synthetic Peptide
HKEGTFTSDVSKYLEEQAAEFIVWLVKGRGKKGGPPPSK

SEQ ID NO: 100 - protein, 39 aa, Artificial Sequence
  Synthetic Peptide
HIEGTFTSDVSKYLEEQAAEFIVWLVKGRGKKGXPPPSK

SEQ ID NO: 101 - protein, 39 aa, Artificial Sequence
  Synthetic Peptide
HVEGTFTSDVSKYLEXQAAEFIVWLVKGRGKKGGPPPSK

SEQ ID NO: 102 - protein, 39 aa, Artificial Sequence
  Synthetic Peptide
HVEGTFTSDVSKYLEXQAAEFIVWLVKGRGKKGXPPPSK

SEQ ID NO: 103 - protein, 39 aa, Artificial Sequence
  Synthetic Peptide
HVEGTFTSDVSKYMEGQAAEFIVWLVKGRGKKGGPPPSK

SEQ ID NO: 104 - protein, 39 aa, Artificial Sequence
  Synthetic Peptide
HIEGTFTSDVSKYMEGQAAEFIVWLVKGRGKKGXPPPSK

SEQ ID NO: 105 - protein, 39 aa, Artificial Sequence
  Synthetic Peptide
HKEGTFTSDVSKYMEEQAAEFIVWLVKGRGKKGGPPPSK

SEQ ID NO: 106 - protein, 39 aa, Artificial Sequence
  Synthetic Peptide
HIEGTFTSDVSKYMEEQAAEFIVWLVKGRGKKGXPPPSK

SEQ ID NO: 107 - protein, 39 aa, Artificial Sequence
  Synthetic Peptide
HKEGTFTSDVSKYMEXQAAEFIVWLVKGRGKKGGPPPSK

SEQ ID NO: 108 - protein, 39 aa, Artificial Sequence
  Synthetic Peptide
HVEGTFTSDVSKYMEXQAAEFIVWLVKGRGKKGXPPPSK

SEQ ID NO: 109 - protein, 39 aa, Artificial Sequence
  Synthetic Peptide
HKEGTFTSDVSKQLEGQAAEFIVWLVKGRGKKGGPPPSK

SEQ ID NO: 110 - protein, 39 aa, Artificial Sequence
  Synthetic Peptide
HIEGTFTSDVSKQLEGQAAEFIVWLVKGRGKKGXPPPSK

SEQ ID NO: 111 - protein, 39 aa, Artificial Sequence
  Synthetic Peptide
HKEGTFTSDVSKQLEEQAAEFIVWLVKGRGKKGGPPPSK

SEQ ID NO: 112 - protein, 39 aa, Artificial Sequence
  Synthetic Peptide
HXEGTFTSDVSKQLEEQAAEFIVWLVKGRGKKGXPPPSK

SEQ ID NO: 113 - protein, 39 aa, Artificial Sequence
  Synthetic Peptide
HKEGTFTSDVSKQLEXQAAEFIVWLVKGRGKKGGPPPSK

SEQ ID NO: 114 - protein, 39 aa, Artificial Sequence
  Synthetic Peptide
HXEGTFTSDVSKQLEXQAAEFIVWLVKGRGKKGXPPPSK

SEQ ID NO: 115 - protein, 39 aa, Artificial Sequence
  Synthetic Peptide
HIEGTFTSDVSKQMEGQAAEFIVWLVKGRGKKGGPPPSK

SEQ ID NO: 116 - protein, 39 aa, Artificial Sequence
  Synthetic Peptide
HXEGTFTSDVSKQMEGQAAEFIVWLVKGRGKKGXPPPSK

SEQ ID NO: 117 - protein, 39 aa, Artificial Sequence
  Synthetic Peptide
HGEGTFTSDVSKQMEEQAAEFIVWLVKGRGKKGGPPPSK

SEQ ID NO: 118 - protein, 39 aa, Artificial Sequence
  Synthetic Peptide
HXEGTFTSDVSKQMEEQAAEFIVWLVKGRGKKGXPPPSK

SEQ ID NO: 119 - protein, 39 aa, Artificial Sequence
  Synthetic Peptide
HKEGTFTSDVSKQMEXQAAEFIVWLVKGRGKKGGPPPSK

SEQ ID NO: 120 - protein, 39 aa, Artificial Sequence
  Synthetic Peptide
HXEGTFTSDVSKQMEXQAAEFIVWLVKGRGKKGXPPPSK

(19) United States
(12) Patent Application Publication
(10) Pub. No.: US 2012/0129768 A1
(43) Pub. Date: May 24, 2012

(54) GLP-1 ANALOGUES AND THEIR PHARMACEUTICAL SALTS AND USES

(75) Inventors: Yali Wang, Lian Yungang (CN); Aifeng Lü, Lian Yungang (CN); Changan Sun, Lian Yungang (CN); Hengli Yuan, Lian Yungang (CN)

(73) Assignee: Jiangsu Hansoh pharmaceutical Group Co. Ltd

(21) Appl. No.: 13/388,056

(22) PCT Filed: Jul. 29, 2010
(86) PCT No.: PCT/CN2010/075548

§ 371 Date: Feb. 9, 2012

(30) Foreign Application Priority Data
Jul. 30, 2009 (CN) 2009101655559.9

(51) Int. Cl. A61K 38/26; A61P 3/10; A61P 3/04; C07K 14/605

(52) U.S. Cl. 514/5.3; 530/308; 514/11.7; 514/7.2

(57) ABSTRACT

This invention discloses GLP-1 analogues and their pharmaceutical salts, wherein the GLP-1 analogue comprises an amino acid sequence of general formula (I), wherein Lys represents a modified lysine with a lipophilic acid. The GLP-1 analogues provided by this invention have the function of human GLP-1, and a longer half-life in vivo compared with the human GLP-1. Uses of such compounds and compositions include treating non-insulin-dependent diabetes, insulin-dependent diabetes, and obesity.

(I)
X₁-X₂-Glu-Gly-Thr-Phe-Thr-Ser-Asp-X₁₀-Ser-X₁₂-
X₁₃-X₁₄-Glu-X₁₆-X₁₇-Ala-X₁₉-X₂₀-X₂₁-Phe-Ile-X₂₄-
Trp-Leu-X₂₇-X₂₈-X₂₉-X₃₀-X₃₁-X₃₂-X₃₃-X₃₄-X₃₅-X₃₆-
X₃₇-X₃₈-X₃₉-Lys

### FIELD OF THE INVENTION

This invention relates to analogues of the human Glucagon-like peptide-1 (GLP-1) and pharmaceutical salts thereof. The GLP-1 analogues provided in this invention have the function of the human GLP-1 peptide and a longer half-life in vivo compared with the native protein. The present invention also relates to the use of GLP-1 analogues, the pharmaceutical salts thereof, or use as a pharmaceutical composition thereof in the treatment of non-insulin-dependent diabetes, insulin-dependent diabetes and obesity.

### BACKGROUND OF THE INVENTION

Diabetes mellitus is a global epidemic disease and is a metabolic disorder relating to glucose, protein and lipids due to the absolute or relative deficiency of insulin (See Chen Ruijie. Status of research on diabetes drugs. Academic journal of Guangdong College of Pharmacy, 2001, 7(2):131-133). Diabetes mellitus can be divided into type I diabetes mellitus and type II diabetes mellitus (Type 2 diabetes mellitus, T2DM, the same below) according to the pathogenesis thereof 90-95% of all the patients diagnosed with diabetes mellitus suffer from T2DM, and patients are often afflicted with obesity, a deficiency of physical activity. T2DM is most common in the aging population, or among those with family history of diabetes mellitus T2DM. It is also a progressive disease. According to statistical data in 2000, the World Health Organization estimated that there are about 171 million people worldwidely who suffer from diabetes mellitus. In 2005, the U.S. Centers for Disease Control and Prevention. estimated that 20.8 million Americans suffer from diabetes mellitus which is about 7% of the population of the United States. In 2006, the International Diabetes Federation, estimated that the global number of patients suffering from diabetes mellitus is about 246 million (about 5.9% of the totally global population) and indicated that 46% of the patients were 40-59 years old.

T2DM is characterized by the inhibition of the secration of insulin and pancreatic β-cell dysfunction which results in insulin deficiency and hyperglycemia. (See Ferrannini E. Insulin resistance versus insulin deficiency in non-insulin-dependent diabetes mellitus: problems and prospects. Endocr Rev. 1998, 19(4):477-490). T2DM patients typically suffer from a postprandial and fasting hyperglycemia (fasting glucose>125 mg/dL). Observed high blood sugar is the result of pancreatic β-cells failure to secrete enough insulin in the surrounding tissue. (See Weyer C., Bogardus C., Mott D M., et al. The natural history of insulin secretory dysfunction and insulin resistance in the pathogenesis of type 2 diabetes mellitus. J. Clin. Invest. 1999, 104(6): 787-794).

A major risk factor of T2DM is obesity, which is itself very harmful to human health. T2DM often co-exists with other high-risk diseases such as hypertension and dyslipidemia. 60% of T2DM patients are accompanied by microvascular complications, including retinopathy and neuropathy, and also are accompanied by cardiovascular morbidities, such as coronary heart disease, myocardial infarction, shock, and the like. In the U.S., cardiovascular diseases (CVD) is the major cause resulting in mortality, and T2DM is the major risk factor causing macrovascular complications such as an atherosclerosis, myocardial infarction, shock, and peripheral vascular diseases. The risk of death caused by heart diseases with diabetes is 2-4 times higher than that of a non-diabetes person. In addition, nearly 65% of people with diabetes die of heart disease.

In addition to the physical and physiological harm to patients, T2DM causes great economic burden on society. According to statistics, the cost of the treatment of complications associated with diabetes is about $ 22.9 billion; the total cost of the treatment of T2DM and complications thereof is nearly $ 57.1 billion every year in the U.S.

Drugs for the treatment of T2DM have been sought. These include the early oral hypoglycemic drugs of sulfonyl class and biguanide class and the recent insulin sensitizer and α-glucosidase inhibitors, the development of animal insulins and human insulins in a variety of new regimes and formulations, the research of new mechanisms of drug treatment by simply increasing insulin, and new ways acting on the insulin-producing cells. Weight gain is a common side-effect after the administration of oral or injection hypoglycemic agents, which may reduce compliance, and may increase the risk of developing cardiovascular disease. Therefore, developing new types of drugs for the treatment of T2DM which have high safety profiles, good patient compliance and low side-effects is desirable.

As early as 100 years ago, Moore proposed that the duodenum can secrete a “chemical stimulant” stimulating pancreatic secretion. Attempts to inject gut-extract to treat diabetes were undertaken. Subsequently it was discovered that humoral factors derived from intestinal secretion can enhance the function of the pancreas endocrine, and about 50% of insulin secretion induced by intravenous or oral glucose is derived from the stimulus of peptides produced in the gut. Therefore Zunz and Labarre described the concept of “incretin.” Two kinds of incretins have been isolated so far, namely glucose-dependent insulinotropic polypeptide (GIP) and glucagon-like peptide-1 (GLP-1). Both GIP and GLP-1 are secreted by specific intestinal nerve cells when a related nutrient is absorbed. GIP is secreted by the duodenum and proximal jejunal K cells. GLP-1 is synthesized in L cells and mainly exists in the distal small bowel and colon (See Drucker D J. Enhancing incretin action for the treatment of type 2 diabetes. Diabetes Care. 2003, 26(10):2929-2940).

GLP-1 exists in two bio-active forms in blood plasma, namely GLP-1 (7-37) and GLP-1 (7-36). The difference between the two forms resides in one amino acid residue, and their biological effects and in vivo half-life are the same. (See Drucker D J. Enhancing incretin action for the treatment of type 2 diabetes. Diabetes Care. 2003, 26(10):2929-2940).

GLP-1 is usually referred to as GLP-1 (7-37) and GLP-1 (7-36) amide. GIP and GLP-1 are degraded to inactive forms by dipeptidyl peptidase-IV(DPP-IV) quickly after released in the gastrointestinal tract, so that the in vivo half-life of GIP and GLP-1 is very short (in vivo half-life of GIP is about 5-7 minutes, in vivo half-life of GLP-1 is about 2 minutes). (See Drucker D J. Enhancing incretin action for the treatment of type 2 diabetes. Diabetes Care. 2003, 26(10):2929-2940). Researches show that most of the degradation process occurrs when the GIP and GLP-1 enter into the blood vessels containing DPP-IV, and a small amount of GLP-1 and GIP which has not been degraded will enter into the pancreas and associate with binding sites to stimulate insulin release from β-cells. Different from the mechanism of sulfonylurea to directly promote functional β-cells to release insulin, most of the effects of incretin are glucose-dependent. In addition, some in vitro tests on animals and humans have shown that GLP-1 also functions to suppress α-cell and reduce glucagon hypersecretion.

Although plasma GIP levels in patients with T2DM are normal, when the function of incretin declines significantly, the GLP-1 levels in patients with T2DM decline. Thus, drugs based on GLP-1 contribute more to treatment of T2DM. Although the levels of both GLP-1 (7-37) and GLP-1 (7-36) amide will increase in several minutes after a meal, and the content of GLP-1 (7-36) amide is more, so the GLP-1 secretion might have been greatly increased by the double effect of endocrine and transmission of neural signal before the digested food enters the small intestine and colon. The plasma level of GLP-1 under a fasting state is very low (about 5-10 pmol/L), and is increased rapidly after eating (up to 15-50 pmol/L). Under the double function of DPP-IV and renal clearance, the level in vivo of GLP-1 in circulation is decreased rapidly. Other enzymes such as human neutral endopeptidase 24·11 may also play a vital role in inactivating clearance of GLP-1. Because the second amino acid residue of GLP-1 is alanine, which is a good substrate of DPP-IV, GLP-1 is easily degraded into inactive peptide fragments. In fact, the DPP-IV in vivo is postulated as the key reason for loss of the activity of the incretin. Experiments show that GLP-1 levels in mice, in which DPP-IV gene has been silenced, is higher than in normal mice. Significantly, insulin secretion is increased, too. Just because the presence of DPP-IV, the content in vivo (except in plasma) of the nondegradaded and biologically active GLP-1 is only 10-20% of the total content of GLP-1 in plasma. (See Deacon C F, Nauck M A, Toft-Nielsen M, et al. Both subcutaneously and intravenously administered, glucagon-like peptide 1 is rapidly degraded from the NH₂-terminus in type 2-diabetic patients and in healthy subjects. (See Diabetes. 1995, 44(9): 1126-1131).

GLP-1 and GIP play their respective roles through binding to different G-protein-coupled receptors (GPCRs). Most of GIP receptors are expressed by pancreatic β-cells, and a minor part of GIP receptors are expressed by adipose tissue and the central nervous system. In contrast, GLP-1 receptors are mainly expressed in the pancreatic α- and β-cells and peripheral tissues including the central and peripheral nervous systems, brain, kidney, lung and gastrointestinal tract and the like. The activation of two incretins in β-cells will result in the rapid increase of the level of cAMP and intracellular calcium, thereby rleading to their extracellular secretion in a glucose-dependent manner. The sustained signal transmission from incretin receptors is associated with protein kinase A, resulting in gene transcription, increasing insulin biosynthesis and stimulating β-cell proliferation. (See Gallwitz B. Glucagon-like peptide-1-based therapies for the treatment of type 2 diabetes mellitus. Treat Endocrinol. 2005, 4(6):361-370). The activation of GLP-1 receptor and GIP receptor can also inhibit the apoptosis of pancreatic β-cells of rodent and human, while increasing their survival (See Li Y, Hansotia T, Yusta B, et al. Glucagon-like peptide-1 receptor signaling modulates beta cell apoptosis. J Biol. Chem. 2003, 278(1): 471-478). Consistent with the expression of GLP-1 receptor, GLP-1 can also inhibit glucagon secretion, gastric emptying and food intake, and enhance the degradation of glucose through the neural mechanism. It shall be noted that, as with other insulin secretion mechanisms, the role of GLP-1 to control the level of glucose is glucagon-dependent and the counter-regulatory release of glucagon caused by low blood sugar is fully retained even at the pharmacological level of GLP-1.

The important physiological role of endogenous GLP-1 and GIP in glucose homeostasis has been studied in-depth through using receptor antagonists or gene knockout mice. Acute antagonism of GLP-1 or GIP reduces insulin secretion in vivo of rodents and increases plasma glucose content. Similarly, the mutant mice, in which GIP or GLP-1 receptor is inactivated, also experience defective glucose-stimulated insulin secretion and damaged glucose tolerance. GLP-1 also has a function of regulating fasting blood glucose, because the acute antagonists or damage on the GLP-1 gene will cause the increase of fasting glucose level of rodents. At the same time, GLP-1 is the basis of glucose control in human bodies, and studies on the antagonist of Exendin (9-39) have shown that the destruction of GLP-1 function will result in defective glucose-stimulated insulin secretion, decreased glucose clearance rate, increased glucagon levels and accelerated gastric emptying. The physiological roles of GLP-1 (see Deacon C F. Therapeutic strategies based on glucagon-like peptide 1. Diabetes. 2004, 53(9):2181-2189) comprise: (1) helping to organize glucose absorption, mediate glucose-dependent insulin secretion; (2) inhibiting postprandial glucagon secretion, reducing hepatic glucose release; (3) regulating gastric emptying, preventing excessive circulating of glucose when the food is absorbed in the intestine; and (4) inhibiting food intake (such as appetite). Also, animal studies also showed a physiological role for stabilizing the number of pancreatic β-cells in vivo.

Due to the beneficial effects of GLP-1 and GIP in controlling blood sugar and many other aspects, especially their characteristics of not producing hypoglycemia and delaying gastric emptying to control weight, the compounds attract the interest of many scientists. Further studies of based on GLP-1 and GIP for the treatment of T2DM have been pursued. It is well known that T2DM patients lack or lose the incretin effect. One reason is that incretin effect of GIP in vivo in the T2DM patient is significantly reduced. Meanwhile, the level of GLP-1 in vivo in T2DM patients is very low, and the level of GLP-1 caused by dietary stimuli is significantly reduced. (See Toft-Nielsen M B, Damholt M B, Madsbad S, et al. Determinants of the impaired secretion of glucagon-like peptide-1 in type 2 diabetic patients. J Clin Endocrinol Metab. 2001, 86(8):3717-3723). Because the role of GLP-1 in vivo in patients with T2DM has been partially reserved, GLP-1 synergist is one of the research directions of the drugs designed to enhance the incretin effect in T2DM patients.

GLP-1 analogues, may act similarly to endogenous GLP, by inhibiting the release of glucagon and stimulate insulin secretion both in vivo in a glucose-dependent manner and thus its role for lowering blood glucose exhibit a self-limitation, which generally does not cause hypoglycemia in large doses. Some literature reports that GLP-1 can reduce blood sugar to a level below normal, and this effect is transient and considered a natural result of GLP-1 promoted insulin secretion. GLP-1 can temporarily reduce blood sugar to a level below normal level but does not cause serious and persistent hypoglycemia. Besides directly reducing blood glucose, GLP-1 can also reduce the quantity of food intake, which has been verified in rodents and humans. The level of blood glucose, therefore, can be controlled by reducing body weight indirectly. GLP-1 also has the potential role of inhibiting the secretion of gastrin and gastric acid stimulated by eating, and these functions show that GLP-1 may also have a role in the prevention of peptic ulcer. Mechanisms of action for GLP-1 make it an ideal drug for the treatment of patients with type 2 diabetes, but also the drug for the treatment of patients with obesity diabetes. GLP-1 can enhance the satiety of the patients, reduce food intake and maintain body weight or lose weight. Several studies suggest that GLP-1 can prevent the conversion from impaired glucose tolerance to diabetes, and some literature reports that the GLP-1 class of compounds has direct effect on the growth and proliferation of pancreatic β-cells in experimental animals. It was found by some experiments that GLP-1 can promote the differentiation from pancreatic stem cells to functional β-cells. These results suggest that GLP-1 has the function of protecting pancreatic islet and delaying the progression of diabetes, and can maintain the morphologies and functions of β-cells, while reduce the apoptosis of β-cells. Because some oral drugs and exogenous insulins can not inhibit or reduce the exorbitant glucagon secretion in patients with T2DM, GLP-1 analogues can affect glucagon hypersecretion through directly inhibiting glucagon release or inhibition of glucagon resulted from promoting insulin secretion. The postprandial hyperglycemia can be reduced effectively through these two mechanisms. Meanwhile, the maintaining of the function of β-cells may also play a role in controlling the long-term postprandial hyperglycemia.

GLP-1 analogues are administered through subcutaneous injection, which doesn't require calculation of the amount of carbohydrates to estimate the optimal drug dosage, and does not require self-monitoring the blood glucose. As a result, these kinds of drugs are easier for patient compliance than self-administered insulin.

A variety of effects of natural GLP-1 have been confirmed, which bring new hope for the treatment of T2DM. The natural human GLP-1 peptide is, however, very unstable and can be degraded by dipeptidyl peptidase IV (DPP-IV). Moreover, its half-life is only about 2 minutes. When using natural GLP-1 to lower blood sugar, continuous intravenous infusion or continuous subcutaneous injection is needed, resulting in its poor clinical feasibility. Faced with this situation, researchers continue to explore methods to extend the action time of GLP-1. Therefore, there is a need for the development of long-acting GLP-1 analogues or derivatives thereof.

Exenatide is a synthetic Exendin-4, which is developed by the Eli Lilly Company and Amylin Company, with the trade name Byetta®. Exenatide has been approved for the treatment of T2DM by FDA and EMEA. It has 50% homology with mammalian GLP-1 in sequence and has a similar affinity site of the receptor with GLP-1. (See Drucker D J, Nauck M A. The incretin system: glucagon-like peptide-1 receptor agonists and dipeptidyl peptidase-4 inhibitors in type 2 diabetes. Lancet. 2006, 368(9548):1696-1705). It is encoded by a lizard-specific gene. Compared with GLP-1, the second residue, alanine, in GLP-1 is replaced with glycine in Exenatide, which effectively inhibits the enzymolysis of DPP-IV enzyme, and its half-life in vivo is about 60-90 minutes. (See Kolterman O G, Kim D D, Shen L, et al. Pharmacokinetics, pharmacodynamics, and safety of exenatide in patients with type 2 diabetes melllitus. Am Health Syst Pharm. 2005, 62(2): 173-181). The in vivo concentration of Exenatide after a single subcutaneous injection is increased persistently and can arrive to the maximum plasma concentration after 2 h or so, which can be maintained for 4-6 hours. (See Nielsen L L, Baron A D. Pharmacology of exenatide (synthetic exendin-4) for the treatment of type 2 diabetes. Curr Opin Investig Drugs. 2003, 4(4):401-05). It should be noted that the metabolism of Exenatide does not occur in the liver, but is degraded mainly by protein protease after filtered by renal glomeruli.

Exenatide has special glucose-regulating activities, including glucose-dependent enhance of insulin secretion, glucose-dependent inhibition of wrong excessive glucagon secretion, slowing gastric emptying and decreasing food intake and the like. Studies in vitro and in vivo in the models of diabetes found that Exenatide also has the effects of storing the first stage (first-phase) insulin secretion, promoting the proliferation of β-cell and promoting the regeneration of insulin from its precursor cell.

In order to achieve better control of blood glucose, injections twice a day of Exenatide are needed. This is a major inconvenience to patients. Furthermore, Exenatide has unfortunate side effects including mild to moderate nausea (about 40% of patients will have this reaction), diarrhea and vomiting (less than 15% of patients have both reactions). In addition, about 50% of Exenatide-treated patients can generate antibodies, although these antibodies do not affect the efficacy or lead to other clinical effects. Recently it is found that six patients suffered hemorrhage or symptoms of necrotizing pancreatitis after taking Byetta®.

CJC-1131 is a GLP-1 analogue with peptidase resistance developed by ConjuChem Biotechnologies Inc., in which the alanine residue in the second position of GLP-1 is replaced with D-Ala to enhance resistance of DPP-IV enzymolysis. The structure contains an active reactive linker that can bind to serum albuminutesthrough a covalent, non-reversible manner. (See Kim J G, Baggio L L, Bridon D P, et al. Development and characterization of a glucagon-like peptide-1 albuminutesconjugate: the ability to activate the glucagon-like peptide 1 receptor in vivo. Diabetes 2003, 52(3):751-759). The GLP-1-serum albuminutescomplex retains the activity of GLP-1, while increasing its stability to DPP-IV enzymolysis, thereby extending in vivo action. Its half-life in plasma is about 20 days.

A study has found that the Ki was approximate 12 nM (the Ki of GLP-1 is 5.2 nM) when CJC-1131-serum albuminutescomplex is bound to Chinese hamster ovary cell transfected with recombinant human pancreatic GLP-1 receptor. Meanwhile the EC₅₀ of the complex activating cAMP is 11-13 nM, wherein the EC₅₀ is similar to GLP-1's EC₅₀. Existing literature reports show that this complex can reduce postprandial blood glucose level of the mice whose blood sugar is normal or high, and tests show that this activity of CJC-1131 acts on a certain functional receptor of GLP-1. Meanwhile in mice, CJC-1131 also has an effect on slowing gastric emptying and inhibiting food intake and the like.

Part of a phase II clinical trial of CJC-1131 has been completed. In September 2005, ConjuChem concluded that CJC-1131 may not be suitable for chronic dosing regimens after analysis of test results and suspended further clinical study

Albugon (albumin-GLP-1) is a long-acting drug for the treatment of T2DM developed by GlaxoSmithKline authorized by Human Genome Sciences Inc., which is a fusion protein of GLP-1 (with mutations increasing the resistance to DDP-IV) and albumin. Its half-life in monkeys is 3 days. The basic idea of the development thereof is to couple the recombinant GLP-1 and serum albuminutes to form a complex, thereby its in vivo half-life is significantly increased. The administration of Albugon effectively reduces blood glucose level of mice, increases insulin secretion, slows gastric emptying and reduces food intake etc. (See Baggio L L, Huang Q, Brown T J, et al. A Recombinant Human Glucagon-Like Peptide (GLP)-1-Albuminutes Protein (Albugon) Mimics Peptidergic Activation of GLP-1Receptor-Dependent Pathways Coupled With Satiety, Gastrointestinal Motility, and Glucose Homeostasis. Diabetes 2004, 53(9):2492-2500). Currently Albugon is in phase III clinical trials.

WO9808871 discloses a GLP-1 derivative which is obtained through the modification on GLP-1 (7-37) with fatty acid. The half life in vivo of GLP-1 is significantly enhanced. WO9943705 discloses a derivative of GLP-1, which is chemically modified at the N-terminus, but some literature reports that modification of the amino acids on the N-terminal will significantly decrease the activity of the entire GLP-1 derivative. (See J. Med. Chem. 2000, 43, 1664 1669). In addition, CN200680006362, CN200680006474, WO2007113205, CN200480004658, CN200810152147 and WO2006097538 etc also disclose a series of GLP-1 analogues or derivatives thereof produced by chemical modification or amino acid substitution, in which the most representative one is liraglutide developed by Novo Nordisk, the phase III clinical trial of which has been finished. Liraglutide is a derivative of GLP-1, whose structure contains a GLP-1 analogue of which the sequence is 97% homologous with human GLP-1, and this GLP-1 analogue is linked with palmitic acid covalently to form Liraglutide, wherein the palmitic acid of the structure of Liraglutide is linked to serum albuminutes non-covalently, and this structural characteristic affects a slower release from the injection site without changing the activity of GLP-1 thereby extending its in vivo half life Meanwhile, the palmitic acid in the structure will form a certain steric hindrance to prevent the degradation by DPP-IV and to reduce renal clearance. Because of the characteristics described above, the half-life of Liraglutide in the human body administered by subcutaneous injection is about 10-14 hours. In theory, it can be administered once on day and the daily dose is 0.6-1.8 mg. On Apr. 23, 2009, Novo Nordisk announced that Committee for Medicinal products for Human Use (CHMP) under the EMEA gave a positive evaluation on Liraglutide and recommended approval of its listing. Novo Nordisk hopes that European Commission would approve its application of listing within two months.

### BRIEF SUMMARY OF THE INVENTION

The present invention describes GLP-1 analogues which have longer half-life in vivo. The GLP-1 analogues described have the same function as that of human GLP-1 and a longer half-life in vivo.

The present invention also includes pharmaceutical compositions comprising GLP-1 analogues and pharmaceutically acceptable salts thereof, for use in the treatment of non-insulin-dependent diabetes mellitus, insulin-dependent diabetes and obesity.

The aims of the present invention are achieved by the following technical solutions. The present invention provides GLP-1 analogues having amino acid sequence of formula (I) or a pharmaceutically acceptable salt thereof:

X1-X2-Glu-Gly-Thr-Phe-Thr-Ser-Asp-X10-Ser-X12-X13-X14-Glu-X16-X17-Ala-X19-X20-X21-Phe-Ile-X24-Trp-Leu-X27-X28-X29-X30-X31-X32-X33-X34-X35-X36-X37-X38-X39-Lys Formula (I)

wherein the GLP-1 analogues contain a lipophilic substituent of formula R₁(CH₂)ₙ—CO—, in which R₁ is selected from CH₃— and HOOC—, n is an integer selected from 8-25, X1, X2, X10, X12, X13, X14, X16, X17, X19, X20, X21, X24, X27, X28, X29, X30, X31, X32, X33, X34, X35, X36, X37, X38 and X39 are independently selected from any natural or non-natural amino acid or the peptide segments consisting of any natural or non natural amino acid.

The GLP-1 analogues refer to a new GLP-1 peptide obtained by the partial amino acid substitution or the extension at the C terminal of human GLP-1 (7-37) peptide serving as a precursor, comprising GLP-1 (7-36) amide and GLP-1 (7-37), which has same function as that of human GLP-1.

The GLP-1 analogues may be modified so that amino acid residues have lipophilic substituents, wherein a typical modification is to form an amide or ester, preferably, to form an amide.

In a preferred embodiment of the invention, the lipophilic substituent of formula R₁(CH₂)ₙ—CO— and the amino group of the amino acid residues of the GLP-1 analogue are linked by an amide bond, in which R₁ is selected from CH₃— and HOOC—, and n is an integer selected from 8-25.

In another preferred embodiment of the invention, the lipophilic substituent of formula R₁(CH₂)ₙ—CO— and the ε amino group of the Lys at the C-terminal of the GLP-1 analogue are linked by an amide bond, in which R₁ is selected from CH₃— and HOOC—, and n is an integer selected from 8-25.

In yet another preferred embodiment of the invention, the lipophilic substituent of formula R_{I}(CH₂)ₙ—CO— and the α amino group of the Lys at the C-terminal of the GLP-1 analogue are linked by an amide bond, in which R₁ is selected from CH₃— and HOOC—, and n is an integer selected from 8-25, and 14 is the most preferred.

In another preferred embodiment of the invention, X1 in the amino acid sequence of the GLP-1 analogue is selected from L-His and D-His; X2 is selected from Ala, D-Ala, Gly, Val, Leu, Ile, Lys and Aib; X10 is selected from Val and Leu; X12 is selected from Ser, Lys and Arg; X13 is selected from Tyr and Gln; X14 is selected from Leu and Met; X16 is selected from Gly, Glu and Aib; X17 is selected from Gln, Glu, Lys and Arg; X19 is selected from Ala and Val; X20 is selected from Lys, Glu and Arg; X21 is selected from Glu and Leu; X24 is selected from Val and Lys; X27 is selected from Val and Lys; X28 is selected from Lys, Glu, Asn and Arg; X29 is selected from Gly and Aib; X30 is selected from Arg, Gly and Lys; X31 is selected from Gly, Ala, Glu, Pro and Lys; X32 is selected from Lys and Ser; X33 is selected from Lys and Ser; X34 is selected from Gly, Ala and Sar; X35 is selected from Gly, Ala and Sar; X36 is selected from Pro and Gly; X37 is selected from Pro and Gly; X38 is selected from Pro and Gly; X39 is selected from Ser and Tyr.

In one more preferred embodiment of the present invention, the amino acid sequence of the GLP-1 analogue is selected from the group consisting of SEQ ID NO: 1 to SEQ ID NO: 120.

In another preferred embodiment of the present invention, the lipophilic substituent of formula R₁(CH₂)ₙ—CO— and the amino group of the amino acid residues of the GLP-1 analog, of which the sequence is selected from the group consisting of SEQ ID NO: 1 to SEQ ID NO: 120, are linked by an amide bond, in which R₁ is selected from CH₃— and HOOC—, and n is an integer selected from 8-25.

In one more preferred embodiment of the present invention, the lipophilic substituent of formula R₁(CH₂)ₙ—CO— and the ε amino group of the C-terminal Lys of the GLP-1 analog, selected from the group consisting of SEQ ID NO: 1 to SEQ ID NO: 120, are linked by an amide bond, in which R₁ is selected from CH₃— and HOOC—, and n is an integer selected from 8-25.

In one more preferred embodiment of the present invention, the lipophilic substituent of formula R₁(CH₂)ₙ—CO— and the α amino group of the C-terminal Lys of the GLP-1 analog, selected from the group consisting of SEQ ID NO: 1 to SEQ ID NO: 120, are linked by an amide bond, in which R₁ is selected from CH₃ and HOOC—, and n is an integer selected from 8-25, preferably n is selected from 8, 10, 12, 14, 16, 18, 20 and 22, most preferably, n is 14.

In one more preferred embodiment of this invention, the lipophilic substituent of formula R₁(CH₂)ₙ—CO— and the α amino group of the C-terminal Lys of the GLP-1 analog, selected from the group consisting of SEQ ID NO: 1 to SEQ ID NO: 20, are linked by an amide bond, in which R₁ is selected from CH₃— and HOOC—, and n is an integer selected from 8-25, preferably n is selected from 8, 10, 12, 14, 16, 18, 20 and 22, most preferably, n is 14.

In another more preferred embodiment of this invention, the lipophilic substituent of formula R₁(CH₂)ₙ—CO— and the α amido of the C-terminal Lys of the GLP-1 analog, selected from the group consisting of SEQ ID NO: 1 to SEQ ID NO: 8, are linked by an amide bond, in which R₁ is CH₃, and n is 14.

The GLP-1 analogues provided in this invention belong to amphoteric compounds and one skilled in the art can convert them into salts by using acid or alkaline compounds with known technologies, wherein acids usually used for the formation of acid addition salts are: hydrochloric acid, hydrobromic acid, hydriodic acid, sulfuric acid, phosphoric acid, p-toluenesulfonic acid, methanesulfonic acid, oxalic acid, p-bromophenyl sulfonic acid, carbonic acid, succinic acid, citric acid, benzoic acid, acetic acid; the salts comprise sulfate, pyrosulfate, trifluoroacetate, sulfite, bisulfite, phosphate, biphosphate, dihydric phosphate, metaphosphate, pyrophosphate, hydrochloride, bromide, iodide, acetate, propionate, octanoates, acrylate, formate, isobutyric acid, hexanoate, enanthates, propiolate, oxalate, malonate, succinate, suberate, fumarate, maleate, 1,4-butynedioate, 1,6-hexynedioate, benzoate, chloro-benzoate, methyl-benzoate, dinitro-benzoate, hydroxyl-benzoate, methoxy-benzoate, phenylacetate, phenpropionate, phenylbutyrate, citrate, lactate, γ-hydroxybutyrate, glycolate, tartrate, methanesulfonate, propanesulfonate, 1-naphthol-sulfonate, 2-naphthol-sulfonate, mandelate and the like, preferably trifluoro-acetate. Alkaline substances can also be turned into salts with GLP-1 analogues, wherein the alkaline substances comprise ammonium, hydroxides of alkali metals or alkaline earth metal, and carbonate, bicarbonate, typically sodium hydroxide, potassium hydroxide, ammonium hydroxide, sodium carbonate, potassium carbonate and the like.

The pharmaceutical compositions containing GLP-1 derivatives according to the invention can be used to treat patients who need this treatment by the way of parenteral administration. Parenteral administration can be chosed from subcutaneous, intramuscular or intravenous injections. The GLP-1 derivatives of the invention can also be administered by transdermal routes, such as administration via transdermal patch (iontophoresis patch and others) and administration through the mucosa.

The pharmaceutical compositions containing the GLP-1 derivatives of the invention can be prepared through common techniques in the art of pharmaceutical industry. These techniques comprise proper dissolving and mixing the components to obtain the desired final compositions. For instance, the GLP-1 derivatives are dissolved in a certain amount of water, wherein the volume of water is slightly less than the final volume of the obtained composition. Isotonic agents, preservatives, surfactants and buffers are added according to need, wherein said isotonic agents are sodium chloride, mannitol, glycerol, propylene glycol, sugar or alditol.

Said preservatives are phenol, orthocresol, para-cresol, meta-cresol, methylparahydroxybenzoate ester, benzyl alcohol. Said appropriate buffering agents are sodium acetate, sodium carbonate, glycine, histidine, lysine, sodium dihydrogen phosphate, disodium hydrogen phosphate, sodium phosphate. Said surfactants are Poloxamer, Poloxamer-188, Poloxamer-407, Tween 80 and Tween-20. If necessary, the aqueous solutions of acids such as hydrochloric acid or alkali such as sodium hydroxide solution are added to adjust pH values of the solutions, and finally the solution volume is adjusted by adding water to obtain the required concentration. Besides said components, the pharmaceutical compositions of the invention also comprise enough basic amino acids or other alkaline reagents having the function to decrease the aggregates formed by the composition during storage, such as lysine, histidine, arginine, imidazole during storage.

The GLP-1 analogues of the invention can be synthesized manually, wherein the resin is HMPA-AM resin, the α-amino group of the amino acid derivatives is protected by the Fmoc (fluorene formyl carbonyl), the side-chain thiol of cysteine, the side-chain amido of glutamine, the side-chain imidazole of histidine are protected by Trt (triphenylmethyl), the side-chain guanidyl of arginine is protected by Pbf (2,2,4,6,7-pentamethyl-dihydrobenzo furan-5-sulfonyl). The side-chain indolyl of tryptophan and the side-chain amino group of lysine are protected by Boc (tert-Butoxycarbonyl) (the side-chain amino group of the Lys are protected by Mtt when the peptide backbone is formed through ε amino group of Lys), the side-chain hydroxyl of threonine, the side-chain phenylol of tyrosine, the side-chain hydroxyl of serine are protected by tBu (tert-butyl). The carboxyl of C-terminal amino acids of the peptide chain of the GLP-1 analogues which will be synthesized is connected with an insoluble high molecular resin (HMP-AM resin) through covalent bonds, and then, the amino acids bound to a solid phase carrier act as amino components, the amino protection group is removed by 20% Hexahydropyridine/DMF solution, and then reactes with excess amino acid derivatives to link a long peptide chain. The operation (Condensation→washing→deprotection→washing→next round of condensation) is repeated to achieve the peptide chain length desired. Finally, the peptide chain is cleaved down from the resins by using mixture of TFA: water: 1,2-dithioglycol: triisopropylsilane(92.5:2.5:2.5:2.5), to obtain the crude GLP-1 analogues through precipitation in an ether. The crude products are purified through using C18 reversed-phase column, and thereby obtaining the desired GLP-1 analogues. The ninhydrin testing method was used to moniter the condensation and the deprotection steps—that is, when there are free aminos on the resin, the ninhydrin reagent will show blue and no color (or slightly yellow) will be shown when there are no free aminos on the resin (Ninhydrin reagent itself is yellow). Therefore, after the condensation reaction is completed, if it shows yellow through ninhydrin test (color of Ninhydrin reagent per se), then it suggests that the coupling step is completed and the deprotection operation before next amino acid coupling can be carried out. If the testing shows blue, it suggests that there are still some free aminos on the peptide chains, and it is needed to further repeat the coupling step or to change the existing condensing agent until the testing has no color or slightly yellow.

### DETAILED DESCRIPTION OF THE INVENTION

To describe the present invention in more detail, the following examples are provided. However, the present invention should not be construed as limited to the embodiments set forth herein.

### Example 1

### The Method for Solid-Phase Synthesis of HS-20001

### 1. Preparation of Fmoc-Lys (Mtt)-HMP-AM Resin

### (1) Drying and Swelling of HMP-AM Resin

50 g (30 mmol) HMP-AM resin (0.6 mmol/g) was dried for 24 hours in vacuum and placed into a 2 L bubbling bottle. Resins were swelled with 500 mL N,N-dimethylformamide (DMF) for 30 minutes, then the DMF was drawn-off and the resins were washed with DMF for 1 minute. The washing step was repeated twice.

### 2. Preparation of Fmoc-Lys(Mtt)-HMP-AM Resin

(1) Coupling of Fmoc-Lys (Mtt)-OH with HMP-AM Resin

The resins were washed with 500 mL DCM and then the washing step was repeated twice. 56.2 g (90 mmol) Fmoc-Lys(Mtt)-OH and 11.4 g (90 mmol) DIC were dissolved in 1 L DCM and added into the swelled HMP-AM resin. 366 mg (3 mmol) DMAP were added to react for 24 hours.

### (2) Washing of the Resin

After the reaction, the resin was washed alternately with DMF and IPA twice and washed with DMF 3 times.

### (3) Capping of Hydroxyl

15.3 g (150 mmol) acetic anhydride and 19.4 g (150 mmol) DIEA were dissolved in 1 L DMF and added into the resin to react for 10 min.

### (4) Washing of the Resin

The resin was washed twice with 1 L 50% MeOH/DMF, 50% DCM/DMF, and then washed three times with DCM and with dehydrated ethanol three times successively. The resin was then dried under vacuum to obtain the Fmoc-Lys(Mtt)-HMP-AM resin.

### (5) Loading Assays of Fmoc-Lys(Mtt)-HMP-AM Resin

5˜10 mg resin were put into 1 mL 20% Hexahydropyridine/DMF solution and stirred for 20 minutes. 50 mL supernatant was removed with a pipet and diluted in 2.5 mL DMF.

Blank samples: 50 mL 20% Hexahydropyridine/DMF was taken with a pipet and is diluted in 2.5 mL DMF.

Degree of substitution is calculated as follows:

Sub=(A×51)/(7.8×m)

wherein A is the absorption value of UV at 301 nm; m is the weight of the resin in mg.

### 2. Swelling of the Solid-Phase Synthesized Resin

50 g (20 mmol) Fmoc-Lys(Mtt)-HMPA-AM resin (0.4 mmol/g) was dried in vacuum for 24 hours and placed into a 2 L bubbling bottle. 500 mL N,N-dimethylformamide (DMF) were added to swell the resin for 30 minutes. The DMF solution was then drawn-off.

### 3. Removal of 4-Methyl Triphenylmethyl (Mtt) Protecting Group of Fmoc-Lys (Mtt)-HMPA-AM Resin

The resin was washed with 200 mL DCM twice followed by addition of 1200 mL 1% TFA/DCM (TFA is about 8-fold excess) to remove Mtt protecting group for 1 hour. The resin was alternately washed with 200 mL 5% N,N-diisopropyl ethylamine (DIEA)/DMF and DMF three times followed by DMF washing three times.

4. Palmitic Acid Condensation 50 mmol palmitic acid and 50 mmol 3-(diethoxyphosphoryloxy)-1,2,3-phentriazine-4-ketone (DEPBT) were dissolved in 400 mL DMF. Then 100 mmol DIEA were added and stirred for 3 minutes at room temperature. The solution was added to the resin, reacted in 37° C. water baths for 2 hours under N₂. After the reaction, the reaction solution was drawn-off and the resin was washed with DMF, isopropyl alcohol (IPA), and DMF in turn.
5. \Removal of 9-Fmoc (Fluorenylmethyloxycarbonyl) Protecting Group of Fmoc-Lys (N-ε-palmitic acid)-HMPA-AM Resin

200 mL 20% piperidine/DMF solution were placed into a bubbling bottle filled with Fmoc-Lys (N-ε-palmitic acid)-HMPA-AM resin and reacted for 5 minutes and then is drawn out. Then 200 mL 20% piperidine/DMF solution were added to react for 20 minutes at room temperature. After the reaction, the resin was washed with 200 mL DMF four times.

### 6. Solid Phase Synthesis of Peptide Chain Part of HS-20001

(1) Condensation of Fmoc-Ser (tBu)-OH

50 mmol Fmoc-Ser(tBu)-OH were dissolved in 125 mL 0.4 M 1-hydroxybenzo triazole (HOBt)/DMF. Then 125 mL 0.4 M N,N′-diisopropyl carbodiimide (DIC)/DCM were added to activate and react for 10 minutes at room temperature. The solution was added into the resin, reacted under of nitrogen at room temperature. Ninhydrin was used to detect and control the degree of the reaction. After reaction, the reaction solution was removed, and the resin was washed with DMF, IPA and DMF in turn.

### (2) Extension of the Peptide Chain

HS-20001 resin peptide was synthesized according to the sequence of the peptide chain of HS-20001 from the amino terminal (N-terminal) to the carboxy-terminal (C-terminal) (His-(D)-Ala-Glu-Gly-Thr-Phe-Thr-Ser-Asp-Leu-Ser-Lys-Gln-Nle-Glu-Glu-Glu-Ala-Val-Arg-Leu-Phe-Ile-Glu-Trp-Leu-Lys-Gln-Gly-Gly-Pro-Ser-Ser-Gly-Ala-Pro-Pro-Pro-Ser), wherein the amounts of amino acids and condensation reagents were the same as the amounts for Fmoc-Ser (tBu)-OH. Protected amino acids were Fmoc-Pro-OH, Fmoc-Ala-OH, Fmoc-Gly-OH, Fmoc-Ser(tBu)-OH, Fmoc-Gln(Trt)-OH, Fmoc-Lys(Boc)-OH, Fmoc-Leu-OH, Fmoc-Trp(Boc)-OH, Fmoc-Glu(OtBu)-OH, Fmoc-Ile-OH, Fmoc-Phe-OH, Fmoc-Arg(Pbf)-OH, Fmoc-Val-OH, Fmoc-Nle-OH, Fmoc-Asp(OtBu)-OH, Fmoc-Thr(tBu)-OH, Fmoc-D-Ala-OH and Fmoc-His(Trt)-OH respectively, and condensation and deprotection reactions were repeated.

### (3) Post-Processing of HS-20001 Resin Peptide

HS-20001 resin peptide obtained in step (2) was washed with DMF, IPA and DMF in turn, then washed with absolute ether twice, and dried under vacuum to obtain the HS-20001 resin peptide.

### (4) Preparation of HS-20001 Crude Peptide

The dried HS-20001 peptide resin is reacted with fresh lysate), of trifluoroacetic acid (TFA): triisopropylsilane (TIS): water=95:2.5:2.5 (by volume and total 10 mL of lysate per gram of the dry resin) for 4 h at room temperature. The reaction solution was filtrated, and the resin was washed with TFA twice. The filtrate was collected, combined, and concentrated to ⅓ of the original volume through rotary evaporation. HS-20001 was precipitated and washed with cold absolute ether, after centrifugation and drying in vacuum, white crude HS-20001 was obtained.

(5) Preparation of HS-20001 with Reversed-Phase Liquid Chromatography

10 g crude HS-20001 was dissolved in a certain amount of water, filtrated with 0.45 μm membrane filter, then purified with reversed-phase high performance liquid chromatography (RP-HPLC), wherein the mobile phase is A 0.1% TFA/H₂O, B 0.1% TFA/acetonitrile,

the column is Denali C-18 column (particle diameter 8.3 μm, 5×30 cm), column temperature is 45° C., detection wavelength is 220 nm, flow rate is 120 mL/min. The product peaks were collected and concentrated under vacuum to remove most of the acetonitrile. 2.25 g of the product (HS-20001) was obtained by lyophilization, of which the purity as 98.5%, and the yield was 22.5%.

### Example 2

### The solid-phase synthesis method for HS-20002

### 1. Preparation of Fmoc-Lys (Mtt)-HMP-AM Resin

See Example 1.

### 2. Swelling of the Solid-Phase Synthesized Resin

50 g (20 mmol) Fmoc-Lys(Mtt)-HMPA-AM resin (0.4 mmol/g) was dried for 24 hours in vacuum and placed into a 2 L bubbling bottle. The resin was swelled with 500 mL DMF for 30 minutes, and then DMF solution was drawn-off

### 3. Removal of Mtt Protecting Group of Fmoc-Lys(Mtt)-HMPA-AM Resin

The resin was washed with 200 mL DCM twice. Mtt protecting group was removed by adding 1200 mL 1% TFA/DCM (TFA is about 8-fold excess) for 1 hour, and then washed with 200 mL 5% DIEA/DMF and DMF alternately for three times followed by DCM washing three times.

### 4. Palmitic Acid Condensation

50 mmol palmitic acid and 50 mmol DEPBT were dissolved in 400 mL DMF, and then 100 mmol DIEA was added by stirring to react for 3 minutes at room temperature. The resulting solution was added to the resin and reacted in 37° C. water bath under N₂ for 2 hours. After the reaction, the reaction solution as removed, and the resin was washed with DMF, isopropyl alcohol (IPA), and DMF in turn.

5. Removal of Fmoc Protecting Group of Fmoc-Lys (N-ε-palmitic acid)-HMPA-AM Resin

200 mL 20% Piperidine/DMF solution was placed into a bubbling bottle filled with Fmoc-Lys(N-ε-palmitic acid)-HMPA-AM resin, and drawn-off after reacting for 5 minutes. 200 mL 20% Piperidine/DMF solution was added for reacting for 20 minutes at room temperature. After the completion, the resin was washed four times with 200 mL DMF.

### 6. Solid-Phase Synthesis Method for the Peptide Chain Part of HS-20002

(1) Condensation of Fmoc-Ser (tBu)-OH

50 mmol Fmoc-Ser(tBu)-OH were dissolved in 125 mL 0.4M HOBt/DMF, then 125 mL 0.4 M DIC/DCM were added to activate and react for 10 minutes at room temperature. The resulting solution was contacted with the resin and reacted under N₂ at room temperature. Ninhydrin was used to detect and control the degree of the reaction. After the reaction, the reaction solution as removed, and the resin was washed with DMF, IPA and DMF in turn.

### (2) Extension of the Peptide Chain

HS-20002 resin peptide was synthesized according to the sequence of peptide chain of HS-20002 from the N-amino (N-terminal) to the carboxy-terminal (C-terminal) (His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser), wherein the amounts of amino acids and condensation reagents were the same as that of Fmoc-Ser (tBu)-OH, protected amino acids were Fmoc-Pro-OH, Fmoc-Ala-OH, Fmoc-Gly-OH, Fmoc-Ser(tBu)-OH, Fmoc-Asn(Trt)-OH, Fmoc-Lys(Boc)-OH, Fmoc-Leu-OH, Fmoc-Trp(Boc)-OH, Fmoc-Glu(OtBu)-OH, Fmoc-Ile-OH, Fmoc-Phe-OH, Fmoc-Arg(Pbf)-OH, Fmoc-Val-OH, Fmoc-Met-OH, Fmoc-Gln(Trt)-OH, Fmoc-Asp(OtBu)-OH, Fmoc-Thr(tBu)-OH and Fmoc-His(Trt)-OH respectively, and condensation and deprotection reactions were repeated.

### (3) Post-Processing of HS-20002 Resin Peptide

The HS-20002 resin peptide obtained in step (2) was washed with DMF, IPA and DMF in turn, then washed twice with absolute ether, then dried under vacuum. HS-20002 resin peptide was obtained therefrom.

### (4) Preparation of HS-20002 Crude Peptide

The dried HS-20002 peptide resin was reacted with fresh lysate of trifluoroacetic acid (TFA): triisopropylsilane (TIS): water: 1,2-ethanedithiol (EDT)=94:1:2.5:2.5 (by volume and total 10 mL of lysate per gram of the dry resin) for 4 hours at room temperature. The reaction solution was filtrated after the reaction. The resin was washed with TFA twice, and then filtrate was collected, combined, and concentrated to ⅓ of the original volume through rotary evaporation. HS-20002 was precipitated with cold absolute ether, after centrifugation and drying under vacuum. The resulting product was white crude HS-20002.

(5) Preparation of HS-20002 with Reversed-Phase Liquid Chromatography

10 g crude HS-20002 were dissolved in a certain amount of water, filtrated with 0.45 μm membrane filter, then purified with reversed-phase high performance liquid chromatography (RP-HPLC), with a mobile phase A was 0.1% TFA/H₂O, B 0.1% TFA/acetonitrile, the column was a Denali C-18 column (particle diameter 8.3 μm, 5×30 cm), column temperature was 45° C., detection wavelength was 220 nm, flow rate was 120 mL/min. The product peaks were collected, concentrated under vacuum to remove most of acetonitrile. 2.1 g of HS-20002 was obtained by lyophilization, of which the purity was 98%, and the yield was 20.5%.

### Example 3

### The Solid-Phase Synthesis Method for HS-20003

### 1. Preparation of Fmoc-Lys (Mtt)-HMP-AM Resin

See Example 1.

### 2. Swelling of the Solid-Phase Synthesized Resin

50 g (20 mmol) Fmoc-Lys(Mtt)-HMPA-AM resin (0.4 mmol/g) dried for 24 hours in vacuum were placed into a 2 L bubbling bottle. The resin was swelled with 500 mL DMF for 30 minutes, and then DMF solution was drawn-off

### 3. Removal of Fmoc Protecting Group of Fmoc-Lys(Mtt)-HMPA-AM Resin

200 mL 20% piperidine/DMF solution were added into a bubbling bottle filled with Fmoc-Lys (Mtt)-HMPA-AM resin. Then the solution was drawn off after 5 minutes, and 200 mL 20% piperidine/DMF solution were added. The reaction continued for another 20 minutes at room temperature. After the reaction, the resin was washed four times with 200 mL DMF.

### 4. Palmitic Acid Condensation

50 mmol Palmitic acid and 50 mmol DEPBT were dissolved in 400 mL DMF. Then 100 mmol DIEA was added by stirring to react for 3 minutes at room temperature. The resulting solution was added to the resin, reacted in 37° C. water baths under N₂ for 2 hours. After the reaction, the reaction solution was removed, and the resin was washed with DMF, isopropyl alcohol (IPA), and DMF in turn.

5. Removal of Mtt Protecting Group of N-ε-Palmitic acid-Lys(Mtt)-HMPA-AM Resin

The resin was washed with 200 mL DCM twice. The Mtt protecting group was removed by adding 1200 mL 1% TFA/DCM (TFA is about 8-fold excess) for 1 hour. The resin was washed with 200 mL 5% DIEA/DMF and DMF alternately three times, then washed with DCM three times.

### 6. Solid-Phase Synthesis Method for the Peptide Chain Part of HS-20003

(1) Condensation of Fmoc-Ser (tBu)-OH

50 mmol Fmoc-Ser(tBu)-OH and 50 mmol DEPBT were dissolved in a certain amount of DCM. Then 100 mmol DIEA was added for activation for 3 minutes at room temperature. The solution was added to the resin, reacted under N₂ at room temperature, and ninhydrin was used to detect and control the degree of the reaction. After the reaction, the reaction solution was removed, and the resin was washed with DMF, IPA and DMF in turn.

### (2) Extension of the Peptide Chain

HS-20003 resin peptide is synthesized according to the sequence of peptide chain of HS-20003 from the N-amino (N-terminal) to the carboxy-terminal (C-terminal) (His-(D)-Ala-Glu-Gly-Thr-Phe-Thr-Ser-Asp-Val-Ser-Ser-Tyr-Leu-Glu-Glu-Glu-Ala-Ala-Lys-Glu-Phe-Ile-Ala-Trp-Leu-Val-Arg-Gly-Gly-Pro-Ser-Ser-Gly-Ala-Pro-Pro-Pro-Ser), wherein the amounts of amino acids and condensation reagents were the same as that of Fmoc-Ser (tBu)-OH, protected amino acids were Fmoc-Pro-OH, Fmoc-Ala-OH, Fmoc-Gly-OH, Fmoc-Ser(tBu)-OH, Fmoc-Arg(Pbf)-OH, Fmoc-Val-OH, Fmoc-Leu-OH, Fmoc-Trp(Boc)-OH, Fmoc-Ile-OH, Fmoc-Phe-OH, Fmoc-Glu(OtBu)-OH, Fmoc-Lys(Boc)-OH, Fmoc-Tyr(tBu)-OH, Fmoc-Asp(OtBu)-OH, Fmoc-Thr(tBu)-OH respectively, and condensation and deprotection reactions were repeated.

### (3) Post-Processing of HS-20003 Resin Peptide

The HS-20003 resin peptide obtained in step (2) was washed with DMF, IPA and DMF in turn, then washed twice with absolute ether, and dried under vacuum to obtain HS-20003 resin peptide.

### (4) Preparation of HS-20003 Crude Peptide

The dried HS-20003 peptide resin was reacted with fresh lysate of trifluoroacetic acid (TFA): triisopropylsilane (TIS): water=95:2.5:2.5 (by volume and total 10 mL of lysate per gram of the dry resin) for 4 hours at room temperature. The reaction solution was filtrated after the reaction. The resin was twice washed with TFA. The filtrate was collected, combined, and concentrated to ⅓ of the original volume through rotary evaporation. HS-20003 was precipitated with cold ether under stirring. After centrifugation and drying in vacuum, white crude HS-20003 was obtained.

(5) Preparation of HS-20003 with Reversed-Phase Liquid Chromatography

10 g crude HS-20003 was dissolved in a certain amount of 20% acetic acid/water and stirred for at least 4 hours, then filtrated with 0.45 μm membrane filter, then purified with reversed-phase high performance liquid chromatography (RP-HPLC), wherein the mobile phase was A 0.1% TFA/H₂O, B 0.1% TFA/acetonitrile, the column was Denali C-18 column (particle diameter 8.3 μm, 5×30 cm), column temperature was 45° C., detection wavelength was 220 nm, flow rate was 120 mL/min. The product peaks were collected, concentrated with vacuum to remove most of acetonitrile. 2.5 g of HS-20003 was obtained by lyophilization, of which the purity was 98.5%, and the yield was 25%.

### Example 4

### The Solid-Phase Synthesis Method for HS-20004

### 1. Preparation of Fmoc-Lys (Mtt)-HMP-AM Resin

See Example 1.

### 2. Swelling of the Solid-Phase Synthesized Resin

50 g (20 mmol) Fmoc-Lys(Mtt)-HMPA-AM resin (0.4 mmol/g) dried for 24 hours under vacuum were placed into a 2 L bubbling bottle. 500 mL DMF was added to swell the resin for 30 minutes, followed by drawing off DMF solution.

### 3. Removal of Fmoc Protecting Group of Fmoc-Lys(Mtt)-HMPA-AM Resin

200 mL 20% piperidine/DMF solution were added into a bubbling bottle filled with Fmoc-Lys (Mtt)-HMPA-AM resin, and then drawn off after 5 minutes, and then 200 mL 20% piperidine/DMF solution was added for reacting for 20 minutes at room temperature. After the reaction, the resin was washed four times with 200 mL DMF.

### 4. Palmitic Acid Condensation

50 mmol palmitic acid and 50 mmol DEPBT were dissolved in 400 mL DMF, and then 100 mmol DIEA was added by stirring for 3 minutes at room temperature. The resulting solution was added to the resin, reacted in 37° C. water bath under N₂ for 2 hours. After the reaction, the reaction solution was removed, and the resin was washed with DMF, isopropyl alcohol (IPA) and DMF in turn.

### 5. Removal of Mtt Protecting Group of Palmitic Acid-Lys(Mtt)-HMPA-AM Resin

The resin was washed with 200 mL DCM twice. Mtt protecting group was removed by adding 1200 mL 1% TFA/DCM (TFA is about 8-fold excess) for reacting for 1 hour, then washed with 5% DIEA/DMF and DMF alternately for three times, then washed three time with DCM.

### 6. The Solid-Phase Synthesis Method for the Peptide Chain Part of HS-20004

(1) Condensation of Fmoc-Ser (tBu)-OH

50 mmol Fmoc-Ser(tBu)-OH and 50 mmol DEPBT were dissolved in a certain amount of DCM. Then 100 mmol DIEA was added for activation for 3 minutes at room temperature. The resulting solution was added to the resin, reacted under N₂ at room temperature, and ninhydrin was used to detect and control the degrees of the reaction. After the reaction, the reaction solution was removed, and the resin was washed with DMF, IPA and DMF in turn.

### (2) Extension of the Peptide Chain

HS-20004 resin peptide was synthesized according to the sequence of the peptide chain of HS-20004 from the N-amino (N-terminal) to the carboxy-terminal (C-terminal) (His-Aib-Glu-Gly-Thr-Phe-Thr-Ser-Asp-Val-Ser-Ser-Tyr-Leu-Glu-Glu-Glu-Ala-Ala-Lys-Glu-Phe-Ile-Ala-Trp-Leu-Val-Arg-Gly-Gly-Pro-Ser-Ser-Gly-Ala-Pro-Pro-Pro-Ser), wherein the amounts of amino acids and condensation reagents were same as that of Fmoc-Ser (tBu)-OH. Protected amino acids were Fmoc-Pro-OH, Fmoc-Ala-OH, Fmoc-Gly-OH, Fmoc-Ser(tBu)-OH, Fmoc-Arg(Pbf)-OH, Fmoc-Val-OH, Fmoc-Leu-OH, Fmoc-Trp(Boc)-OH, Fmoc-Ile-OH, Fmoc-Phe-OH, Fmoc-Glu(OtBu)-OH, Fmoc-Lys(Boc)-OH, Fmoc-Tyr(tBu)-OH, Fmoc-Asp(OtBu)-OH, Fmoc-Thr(tBu)-OH, Fmoc-Aib-OH and Fmoc-His(Trt)-OH respectively, and the condensation and deprotection reactions were repeated.

### (3) Post-Processing for HS-20004 Resin Peptide

HS-20004 resin peptide obtained in step (2) was washed with DMF, IPA and DMF in turn, then washed twice with absolute ether, followed by drying under vacuum to obtain HS-20004 resin peptide.

### (4) Preparation of Crude HS-20004 Peptide

The dried HS-20004 resin peptide was reacted with fresh lysate of trifluoroacetic acid (TFA): triisopropylsilane (TIS): water=95:2.5:2.5 (by volume and total 10 mL of lysate per gram of the dry resin) for 4 hours at room temperature. The reaction solution was filtrated, and the resin was washed twice with TFA. The filtrate was collected, combined, and concentrated to ⅓ of the original volume through rotary evaporation. HS-20004 was precipitated with cold ether under stirring. After centrifugation and drying in vacuum, white crude HS-20004 was obtained.

(5) Preparation of HS-20004 with Reversed-Phase Liquid Chromatography

10 g crude HS-20002 was dissolved in a certain amount of 20% acetic acid/water and stirred for at least 4 hours, then filtrated with 0.45 μm membrane filter, and purified with reversed-phase high performance liquid chromatography (RP-HPLC), wherein mobile phase was A 0.1% TFA/H₂O, B 0.1% TFA/acetonitrile, the column was Denali C-18 column (particle diameter 8.3 μm, 5×30 cm), column temperature was 45° C., detection wavelength was 220 nm, flow rate was 120 mL/min. The product peaks were collected, concentrated with under vacuum to remove most of acetonitrile. 2.25 g of HS-20004 was obtained by lyophilization, of which the purity was 98.5%, and the yield was 22.5%.

### Example 5

### The Solid-Phase Synthesis Method for HS-20005

The preparation method of HS-20005 is as same as that described in example 4, wherein the difference is that the amino acid sequence is replaced with SEQ ID NO: 5, and 2.5 g HS-20005 product was obtained, the purity of which was 98.5%, and the yield was 25%.

### Example 6

### The Solid-Phase Synthesis Method for HS-20006

The preparation method of HS-20006 was the same as that described in example 4, wherein the difference was that the amino acid sequence was replaced with SEQ ID NO: 6. 2.25 g of HS-20006 product was obtained, the purity of which is 98.5%, and the yield is 22.5%.

### Example 7

### The Solid-Phase Synthesis Method for HS-20007

The preparation method of HS-20007 was the same as that described in example 4, wherein the difference is that the amino acid sequence was replaced with SEQ ID NO: 7. 2.1 g of HS-20007 product was obtained, the purity of which was 98%, and the yield was 20.5%.

### Example 8

### The Solid-Phase Synthesis Method for HS-20008

The preparation method of HS-20008 was the same as that described in example 4, wherein the difference is that the amino acid sequence was replaced with SEQ ID NO: 8. 2.5 g of HS-20008 product was obtained, the purity of which was 98.5%, and the yield was 25%.

### Reference Example Solid-Phase Synthesis Method for Liraglutide

### 1. Preparation of Fmoc-Lys(Mtt)-HMP-AM Resin

(1) Drying and swelling of HMP-AM resin

50 g (30 mmol) HMP-AM resin (0.6 mmol/g) was dried for 24 hours in vacuum and placed into a 2 L bubbling bottle. 500 mL N,N-dimethylformamide (DMF) was added to swell therein for 30 minutes. The DMF solution was drawn-off, and DMF was added to wash the resin for 1 minute. This washing step was repeated twice.

### (2) Preparation of Fmoc-Lys(Mtt)-HMP-AM Resin

\textcircled{1} Coupling of Fmoc-Lys (Mtt)-OH and HMP-AM Resin

The resin was washed three times with 500 mL DCM/56.2 g (90 mmol) Fmoc-Lys(Mtt)-OH and 11.4 g (90 mmol) DIC were dissolved in 1 L DCM, and then added into the swelled HMP-AM resin. 366 mg (3 mmol) DMAP was added and reaction proceeded for 24 hours.

\textcircled{2} Washing of the Resin

After the reaction, the resin was washed twice alternately with DMF and IPA and then washed three times with DMF.

\textcircled{3} Capping of Hydroxyl

15.3 g (150 mmol) acetic anhydride and 19.4 g (150 mmol) DIEA were dissolved in 1 L DMF and added to the resin for reacting for 10 minutes.

\textcircled{4} Washing of the Resin

The resin was washed twice with 1 L 50% MeOH/DMF, 50% DCM/DMF, three times with DCM, and was washed three times with absolute ethanol. It was then dried under vacuum to obtain the Fmoc-Lys(Mtt)-HMP-AM resin.

(3) Loading assays of Fmoc-Lys(Mtt)-HMP-AM Resin
5˜10 mg resin were put into 1 mL 20% Hexahydropyridine/DMF solution and stirred for 20 minutes. 50 μL supernatant is taken with a pipet and diluted in 2.5 mL DMF.
Blank samples: 50 μL 20% Hexahydropyridine/DMF was taken with a pipet and is diluted in 2.5 mL DMF.
Degree of substitution is calculated as follows:

Sub=(A×51)/(7.8×m)

wherein A is the absorption value of UV at 301 nm; m is the weight of the resin in mg.

### 2. Swelling the Resin of the Solid-Phase Synthesis

50 g (20 mmol) Fmoc-Gly-HMP-AM resin (0.4 mmol/g) dried for 24 hours in vacuum were placed into a 2 L bubbling bottle, and then 500 mL N,N-dimethylformamide (DMF) was added to swell the resin for 30 minutes. Thereafter, the DMF solution was drawn-off.

### 3. The Solid Phase Synthesis Method of the Peptide Chain Part of Liraglutide

\textcircled{1} condensation of Fmoc-Arg(Pbf)-OH

50 mmol Fmoc-Arg(Pbf)-OH were dissolved in 125 mL 0.4M 1-hydroxybenzotriazole (HOBt)/DMF, then 125 mL 0.4M N,N′-diisopropylcarbodiimide (DIC)/DCM were added to activate and react for 10 minutes at room temperature. The resulting solution was added to the resin, reacted under N₂ at room temperature, and ninhydrin was used to detect and control the degrees of the reaction. After the reaction, the reaction solution was drawn off, and the resin was washed with DMF, IPA and DMF in turn.

\textcircled{2} Extension of the Peptide Chain

Precursor peptide of Liraglutide was synthesized according to the sequence of the peptide chain of Liraglutide from the N-amido (N-terminal) to the carboxy-terminal (C-terminal) (His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly), wherein the amounts of amino acids and condensation reagents were the same as that of Fmoc-Arg(Pbf)-OH, protected amino acids were Fmoc-Arg(Pbf)-OH, Fmoc-Val-OH, Fmoc-Leu-OH, Fmoc-Trp(Boc)-OH, Fmoc-Ala-OH, Fmoc-Ile-OH, Fmoc-Phe-OH, Fmoc-Glu(OtBu)-OH, Fmoc-Lys(Mtt)-OH, Fmoc-Gln(Trt)-OH, Fmoc-Tyr(tBu)-OH, Fmoc-Ser(tBu)-OH, Fmoc-Asp(OtBu)-OH, Fmoc-Thr(tBu)-OH, Fmoc-His(Trt)-OH respectively, and the condensation and deprotection reactions were repeated.

\textcircled{3} Removal of Mtt Protecting Group of the Precursor Peptide of Liraglutide

The resin was twice washed with 200 mL DCM. The Mtt protecting group was removed twice by adding 1200 mL 1% TFA/DCM (TFA is about 8-fold excess) to react for 1 hour. The resin was washed alternately with 200 mL 5% N,N-diisopropylethylamine (DIEA)/DMF and DMF for three times, and washed 3 times with DMF.

\textcircled{4} Modification of Precursor Peptide of Liraglutide with Palmitic Acid

50 mmol Fmoc-Glu-OtBu were dissolved in 125 mL 0.4 M 1-hydroxybenzo triazole (HOBt)/DMF. Then 125 mL 0.4M N,N′-diisopropylcarbodiimide (DIC)/DCM was added to activate and react for 10 minutes at room temperature. The solution was added to the resin from step \textcircled{3}, and allowed to react under N₂ at room temperature.

Ninhydrin was used to detect and control the degree of the reaction or reaction progress. After the reaction, the reaction solution was drawn-off, and the resin is washed with DMF, IPA and DMF in turn.

1 L 20% PIP/DMF was added to remove Fmoc protecting group for 5 minutes, then drawn off 1 L of 20% PIP/DMF was added to remove Fmoc protecting group for 20 minutes, then are drawn off The resulting resin was washed four times with DMF.

50 mmol palmitic acid and 50 mmol 3-(diethoxyphosphoryloxy)-1,2,3-phentriazine-4-ketone (DEPBT) was dissolved in 400 mL DMF. Then 100 mmol DIEA was added to react for 3 minutes under stirring at room temperature. The solution was added to the resin, reacted in 37° C. water bath under N₂ for 2 hours. After the reaction, the reaction solution was drawn-off, and the resin was washed with DMF, isopropyl alcohol (IPA), and DMF in turn.

### 4. Post-Processing of the Resin Peptide of Liraglutide

The resin peptide of Liraglutide obtained in step (2) was washed with DMF, IPA and DMF in turn, and then washed three times with DCM, washed twice with absolute ether, and dried in vacuum, to give the resin peptide of Liraglutide.

### 5. Preparation of Crude Peptide of Liraglutide

The dried peptide resin of Liraglutide was reacted with fresh lysate of trifluoroacetic acid (TFA): triisopropylsilane (TIS): water=95:2.5:2.5 (by volume and total 10 mL of lysate per gram of the dry resin) for 4 hours at room temperature. The reaction solution was filtrated after the reaction, and the resin was twice washed with TFA. The filtrate was collected, combined, and concentrated to ⅓ of the original volume through rotary evaporation. Liraglutide was precipitated with cold absolute ether, after centrifugation and drying under vacuum as white crude HS-20001 is obtained.

\textcircled{5} Preparation of Liraglutide with Reversed-Phase Liquid Chromatography

10 g of crude Liraglutide as dissolved in a certain amount of NH₄HCO₃ solution, filtrated with 0.45 μm membrane filter, then purified with reverse-phase high performance liquid chromotagraphy (RP-HPLC), wherein mobile phase was A 0.1% TFA/H₂O, B 0.1% TFA/acetonitrile, the column was Denali C-18 column (particle diameter 8.3 μm, 5×30 cm), column temperature was 45° C., detection wavelength was 220 nm, flow rate was 120 mL/min. The product peaks were collected, concentrated with vacuum to remove most of acetonitrile. 2.25 g of Liraglutide product was obtained by lyophilization, the purity of which was 98%, and the yield was 12.5%.

### Experimental Example 1

### Testing the Agonist Activity of the Compounds on Glucagon-Like Peptide-1 Receptor (GLP1R)

GLP1R is a receptor coupled with Gs protein, of which the binding with the agonists will result in an increase of intracellular cAMP concentration. In the present experiment, GLP1R and the luciferase reporter gene plasmid regulated by cAMP response elements are co-transfected into HEK293 cells. When the compound binds to the receptor and activates the receptors, the expression of the luciferase will increase. The activation status of the compound to GLP1R can be learned by testing the activity of the luciferase.

| No. of the test | | | Final concentration |
|---|---|---|---|
| drug: | amount (mg) | DMSO (μl) | (mM) |
| Liraglutide | 2 | 53.31556 | 10 |
| HS-20001 | 2 | 43.81871 | 10 |
| HS-20002 | 2 | 43.91502 | 10 |
| HS-20003 | 2 | 44.99387 | 10 |
| HS-20004 | 2 | 44.8526 | 10 |
| HS-20005 | 2 | 43.81871 | 10 |
| HS-20006 | 2 | 43.91502 | 10 |
| HS-20007 | 2 | 44.99387 | 10 |
| HS-20008 | 2 | 44.8526 | 10 |

### Experimental Procedures

1. HEK293 cells stably transfected with GLP1R and pCRE-Luc plasmid were implanted in 96 well plate with the amount 40000 cells/well/100 μl, and incubated at 37° C. for 24 hours.
2. The compounds or positive drugs having a certain concentration gradient were added (3 wells per concentration) and incubated at 37° C. for 5 hours. The negative control was solvent DMSO.
3. 50 μl culture medium was taken from each well, and 50 μl of the luciferase substrate were added and vortexed for 10 minutes.
4. 80 μl reaction solution was taken and transferred to a white 96 well plate, then detected on the Invision microplate reader (enzyme-labelling measuring instrument).

The experimental results: compared with positive compounds liraglutide, the activity of the compound HS-20001 is approximately equal to that of the positive compounds, but HS-20002-20008 show much better agonist activity.

TABLE 1
EC50 Values of the series of compounds:

| | | | Maximum |
|---|---|---|---|
| Compounds | EC50 (nM) | 95% CI (nM) | reaction rate (%) |
| Liraglutide | 0.014707 | 9.726e−012 to | 96.84616 |
| | | 2.223e−011 | |
| HS-20001 | 0.013552 | 7.6757e−012 to | 98.11013 |
| | | 2.3963e−011 | |
| HS-20002 | 0.0014145 | 1.2036e−012 to | 87.99447 |
| | | 1.6623e−012 | |
| HS-20003 | 0.00071876 | 4.9657e−013 to | 87.86082 |
| | | 1.0404e−012 | |
| HS-20004 | 0.00037259 | 2.1453e−013 to | 90.81368 |
| | | 6.4710e−013 | |
| HS-20005 | 0.00023552 | 7.3567e−012 to | 89.13468 |
| | | 2.2346e−011 | |
| HS-20006 | 0.00064358 | 1.3581e−012 to | 87.4281 |
| | | 1.4523e−012 | |
| HS-20007 | 0.00054921 | 4.1354e−013 to | 87.0389 |
| | | 1.2514e−012 | |
| HS-20008 | 0.00021002 | 2.2436e−013 to | 88.4628 |
| | | 6.0245e−013 | |

### Experimental Example 2

### Test In Vivo Activity

db/db mice with type 2 diabetes were divided into six groups based on a random blood glucose and body weight (8 per group). Physiological saline, 3 or 10 μg/kg HS series new compounds (Liraglutide, 20001, 20002, 20003, 20004, 20005, 20006, 20007, 20008) are administered by single subcutaneous injection. The random blood glucose of the mice is determined at different time after administration.

The animals used in the experiment are db/db mice, which are products of a U.S. corporation named Jackson and are conserved and reproduced by Shanghai Institute of Materia Medica of Chinese Academy of Science, of which the Certificate of Conformity is: SCXK(HU)2008-0017, Body Weight: 35-50 g; Gender: Male 85, female 86, bred in SPF-grade animal room; Temperature: 22-24° C.; Humidity: 45-80%; Light: 150-300 Lx, 12 h day alternates with night.

The test candidates of the experiment are HS-20001, HS-20002, HS-20003, HS-20004, HS-20005, HS-20006, HS-20007, HS-20008, liraglutide (developed by Novo Nordisk, as Positive control).

Preparation method: 1 bottle of the compound (2 mg/bottle) was dissolved with double-distilled water to prepare a colorless and transparent solution of which the concentration is 2 mg/mL. Then the solution was diluted to 0.6 μg/mL and 2 μg/mL with physiological saline (Sodium chloride injection, Double-Crane Pharmaceutical Co., Ltd. Anhui, batch number: 080728 6C). “ACCU-CHEK® Advantage” blood glucose meter form Roche was used to determine the blood glucose.

### Dose Setting and Group

### Test Group 1:

Control group: physiological saline
Liraglutide group 3 μg/kg
HS-20001group: 3 μg/kg
HS-20002group: 3 μg/kg
HS-20003group: 3 μg/kg
HS-20004group: 3 μg/kg
HS-20005group: 3 μg/kg
HS-20006group: 3 μg/kg
HS-20007group: 3 μg/kg
HS-20008group: 3 μg/kg

### Test Group 2:

### Control Group: Physiological Saline

Liraglutide group: 10 μg/kg
HS-20001group: 10 μg/kg
HS-20002group: 10 μg/kg
HS-20003 group: 10 μg/kg
HS-20004group: 10 μg/kg
HS-20005group: 10 μg/kg
HS-20006group: 10 μg/kg
HS-20007group: 10 μg/kg
HS-20008group: 10 μg/kg

Route and volume of administration: Single subcutaneous injection dose, dose volume was 5 mL/kg.

### Test Method

Screening, grouping, and administration for db/db mice with type 2 diabetes

### Test Group 1:

171 db/db mice (male 85, female 86) were single-cage reared after weaning, fed with high fat diet. The random and fasting blood glucoses were measured after the db/db mice were seven weeks old. 80 db/db mice which fall ill were picked out and divided into 10 groups according to random blood glucose. Fasting blood glucose and body weight as follows: model control group, Liraglutide group-3 μg/kg, HS-20001 group-3 μg/kg, HS-20002 group-3 μg/kg, HS-20003 group-3 μg/kg, HS-20004 group-3 μg/kg, HS-20005 group-3 μg/kg, HS-20006 group-3 μg/kg, HS-20007 group-3 μg/kg and HS-20008 group-3 μg/kg.

### Test Group 2:

The random blood glucoses of db/db mice were measured. 80 db/db mice which fall ill were picked out and are divided into 10 groups according to random blood glucose and body weight as follows: model control group, Liraglutide group-10 μg/kg, HS-20001 group-10 μg/kg, HS-20002 group-10 μg/kg, HS-20003 group-10 μg/kg, HS-20004 group-10 μg/kg, HS-20005 group-10 μg/kg, HS-20006 group-10 μg/kg, HS-20007 group-10 μg/kg and HS-20008 group-10 μg/kg.

Each group has 8 mice, half male and half female. The animals of each group were administered with the test compounds or solvent control respectively through single subcutaneous injection. The random blood glucose was determined at 1 h, 2 h, 4 h, 8 h and 24 h after administration, and the decrease rate of blood glucose as calculated as follows:

Decrease rate of blood glucose=(blood glucose of control group-blood glucose of treatment group)/blood glucose of control group*100%.

### The Experimental Results

Test 1: Effect of the Low-Dose New Compounds Administered by Singe Dose on Random Blood Glucose of Db/Db Mice

The results can be seen in Tables 2 and 3. db/db mice were administered with 3 μg/kg HS-20002, 20004, 20005, 20006, 20007, or 20008 through single subcutaneous injection. After one hour, random blood glucose values of the mice were decreased significantly compared with those of the control group (P<0.05). Decrease rates are 24.51%, 15.00%, 14.00%, 14.25%, 13.98% and 13.90% respectively. After 2 h and 4 h from administration, random blood glucose values kept a lower level and had significant difference from those of the control group (P<0.05). After 8 hours from administration, random blood glucose values had no significant difference from those of the control group. The mice were administered with 3 μg/kg HS-20003 through subcutaneous injection. After one hour, random blood glucose values were decreased significantly compared with those of the control group (P<0.05); up to 17.33%, after 2 h, 4 h and 8 h from the administration, random blood glucose values showed no significant difference from those of the control group. After administered with 3 μg/kg HS-20001 for db/db mice through single subcutaneous injection, random blood glucose values decreased and were compared to those of the control group No significant difference was observed. The values of random blood glucose of the group of mice administered with liraglutide had no significant decrease.

TABLE 2
Effect of the administration of the new compounds (mmol/L, X̅ ±
s, n = 8) through single dose on random blood glucose of db/db mice in the same day.

| | Before | Time after administration (h) | | | |
|---|---|---|---|---|---|
| groups | administration | 1 | 2 | 4 | 8 |
| control | 25.14 ± 1.09 | 23.66 ± 0.73 | 22.63 ± 0.97 | 22.00 ± 1.00 | 25.39 ± 1.08 |
| Liraglutide—3 μg/kg | 25.11 ± 2.33 | 21.78 ± 2.31 | 23.15 ± 2.62 | 21.56 ± 137 | 23.93 ± 2.09 |
| HS-20001—3 μg/kg | 25.21 ± 1.44 | 20.34 ± 2.29 | 19.84 ± 1.76 | 20.74 ± 2.51 | 24.29 ± 1.60 |
| HS-20002—3 μg/kg | 25.25 ± 1.57 | 17.86 ± 1.90* | 19.56 ± 0.90* | 18.10 ± 0.79** | 24.19 ± 1.79 |
| HS-20003—3 μg/kg | 25.16 ± 1.49 | 19.56 ± 1.19* | 19.44 ± 1.48 | 19.63 ± 1.12 | 22.59 ± 1.05 |
| HS-20004—3 μg/kg | 25.11 ± 1.63 | 20.11 ± 1.28* | 18.81 ± 1.50* | 17.98 ± 1.38* | 23.30 ± 1.47 |
| HS-20005—3 μg/kg | 25.21 ± 1.56 | 20.11 ± 1.19* | 18.96 ± 1.50* | 18.98 ± 1.48* | 22.36 ± 1.67 |
| HS-20006—3 μg/kg | 25.11 ± 1.49 | 20.36 ± 1.25* | 19.91 ± 1.70* | 19.58 ± 1.54* | 24.30 ± 1.50 |
| HS-20007—3 μg/kg | 25.16 ± 1.63 | 20.43 ± 1.19* | 19.81 ± 1.610* | 20.98 ± 2.38* | 23.42 ± 1.38 |
| HS-20008—3 μg/kg | 25.11 ± 1.58 | 20.56 ± 1.30* | 20.81 ± 1.70* | 19.30 ± 2.02* | 22.41 ± 1.51 |

*P < 0.05,
**P < 0.01, Compared with those of the control group

TABLE 3
The decrease rate of random blood glucose (%,
n = 8) of db/db mice administered with the
new compounds through single dose in the same day

| | Time after administration (h) | | | |
|---|---|---|---|---|
| group | 1 | 2 | 4 | 8 |
| Liraglutide—3 μg/kg | 7.98% | −2.32% | 1.99% | 5.76% |
| HS-20001—3 μg/kg | 14.05% | 12.32% | 5.74% | 4.33% |
| HS-20002—3 μg/kg | 24.51% | 13.54% | 17.73% | 4.73% |
| HS-20003—3 μg/kg | 17.33% | 14.09% | 10.80% | 11.03% |
| HS-20004—3 μg/kg | 15.00% | 16.85% | 18.30% | 8.22% |
| HS-20005—3 μg/kg | 14.00% | 12.87% | 10.53% | 8.02% |
| HS-20006—3 μg/kg | 14.25% | 13.12% | 10.86% | 8.14% |
| HS-20007—3 μg/kg | 13.98% | 11.85% | 9.30% | 6.54% |
| HS-20008—3 μg/kg | 13.90% | 11.62% | 8.90% | 6.25% |

Test 2: Effect of the High-Dose New Compounds Administered by Single Dose on Random Blood Glucose of Db/Db Mice

The results can be seen in Tables 4 and 5. db/db mice were administered with 10 μg/kg HS-20002 through single subcutaneous injection. After one hour, the random blood glucose values of the mice decreased significantly compared with those of the control group (P<0.01). After 2 h, 4 h and 8 h from the administration, the random blood glucose values kept a lower level, wherein the values at 4 h after administration were most obvious, of which the decrease rate is up to 40.67% and is significantly different from that of the control group (P<0.001), until 24 hours after administration, the random blood glucose values were still significantly lower than those of the control group. The mice were administered with 10 μg/kg HS-20003 through single subcutaneous injection. After one hour, the random blood glucose values were decreased significantly compared with those of the control group (P<0.01) and is up to 23.62% decreasing, after 2 h, 4 h and 8 h from the administration. The random blood glucose values still keep at a lower level. After 24 hours from administration, there was no significant difference compared with the control group. db/db mice are administered with 10 μg/kg HS-20001 through single subcutaneous injection, after 2 h, the random blood glucose values are decreased significantly compared with those of the control group, after 4 h and 8 h from the administration, the random blood glucose values still keep at a lower level. After 24 hours from administration, the random blood glucose values showed no significant difference from those of the control group. HS-20002, HS-20004, HS-20005, HS-20006, HS-20007 or HS-20008 were administered to mice through single subcutaneous injection and the random blood glucose values are decreased immediately and significantly. The decrease rate is up to 36.20%, after 2 hours. After 4 and 8 hours from the administration, the blood glucose values still kept at a lower level. After 24 hours from the administration, blood glucose was not significantly different compared with those of the control group. The values of random blood glucose of mice of group administered with liraglutide have no significant decrease.

TABLE 4
Effect of the new compounds administered through single dose
(mmol/L, ±s, n = 8) on the random blood glucose of db/db mice in the same day.

| | Before | Time after administration (h) | | | | |
|---|---|---|---|---|---|---|
| | administration | 1 | 2 | 4 | 8 | 24 |
| Group | | | | | | |
| Control | 23.08 ± 1.37 | 27.15 ± 1.51 | 28.49 ± 1.58 | 30.76 ± 1.15 | 29.96 ± 0.88 | 27.75 ± 1.64 |
| liraglutide-10 μg/kg | 23.19 ± 1.35 | 28.59 ± 1.50 | 28.89 ± 1.17 | 28.55 ± 1.31 | 31.84 ± 0.65 | 27.78 ± 1.14 |
| HS-20001-10 μg/kg | 23.16 ± 1.57 | 23.90 ± 1.79 | 20.94 ± 1.57** | 20.20 ± 1.78*** | 23.86 ± 1.87* | 24.60 ± 1.92 |
| HS-20002-10 μg/kg | 23.15 ± 1.32 | 19.74 ± 1.16** | 20.31 ± 2.01** | 18.25 ± 1.98*** | 22.55 ± 2.20** | 22.60 ± 1.46* |
| HS-20003-10 μg/kg | 23.20 ± 1.36 | 20.74 ± 0.98** | 21.10 ± 0.80*** | 19.54 ± 1.80*** | 22.14 ± 2.16** | 24.45 ± 1.55 |
| group | | | | | | |
| control | 23.08 ± 1.37 | 30.76 ± 1.15 | 30.29 ± 0.98 | 29.90 ± 0.89 | 31.04 ± 0.94 | 28.98 ± 1.62 |
| HS-20004-10 μg/kg | 23.18 ± 1.65 | 19.63 ± 1.81*** | 21.86 ± 1.66*** | 21.44 ± 1.68*** | 23.80 ± 1.46*** | 25.64 ± 1.85 |
| HS-20005-10 μg/kg | 23.64 ± 1.35 | 19.39 ± 1.61*** | 21.56 ± 1.56*** | 21.49 ± 1.34*** | 23.46 ± 1.51*** | 25.52 ± 1.68 |
| HS-20006-10 μg/kg | 23.54 ± 1.39 | 19.52 ± 1.72*** | 21.43 ± 1.49*** | 21.53 ± 1.67*** | 23.39 ± 1.55*** | 25.59 ± 1.74 |
| HS-20007-10 μg/kg | 23.56 ± 1.42 | 19.41 ± 1.54*** | 21.84 ± 1.57*** | 21.64 ± 1.56*** | 23.81 ± 1.67*** | 25.51 ± 1.53 |
| HS-20008-10 μg/kg | 23.49 ± 1.49 | 19.38 ± 1.83*** | 21.61 ± 1.68*** | 21.72 ± 1.63*** | 23.56 ± 1.80*** | 25.72 ± 1.69 |

*P < 0.05,
**P < 0.01,
***P < 0.001, Compared with the control group

TABLE 5
The decrease rate of the random blood glucose (%,
n = 8) of db/db mice administered with the
new compounds through single dose in the same day.

| | Time after administration (h) | | | | |
|---|---|---|---|---|---|
| group | 1 | 2 | 4 | 8 | 24 |
| liraglutide—10 μg/kg | −5.29% | −1.40% | 7.19% | −6.26% | −0.09% |
| HS-20001—10 μg/kg | 11.97% | 26.50% | 34.34% | 20.36% | 11.35% |
| HS-20002—10 μg/kg | 27.30% | 28.71% | 40.67% | 24.74% | 18.56% |
| HS-20003—10 μg/kg | 23.62% | 25.93% | 36.49% | 26.12% | 11.89% |
| HS-20004—10 μg/kg | 36.20% | 27.82% | 28.30% | 23.32% | 11.52% |
| HS-20005—10 μg/kg | 37.58% | 28.32% | 29.12% | 24.10% | 12.46% |
| HS-20006—10 μg/kg | 38.12% | 27.66% | 29.78% | 23.72% | 13.66% |
| HS-20007—10 μg/kg | 36.72% | 25.43% | 26.54% | 23.03% | 12.16% |
| HS-20008—10 μg/kg | 35.49% | 25.79% | 27.33% | 22.57% | 14.58% |

Test Conclusions:

The random blood glucose of db/db mice administered with series of the new compounds of the invention through single subcutaneous injection decreased significantly. The random blood glucose level decreased obviously by HS-20002, HS-20003, HS-20004, HS-20005, HS-20006, HS-20007 and HS-20008 in a dose of 3 μg/kg. Where, HS-20002 and HS-20004 show a much better effect on reducing random blood glucose, the duration of the hypoglycemic effect after single subcutaneous injection was dose-related. The duration of the effect of HS-20002 and HS-20004 on decreasing random blood glucose in the dose of 3 μg/kg was more than 4 hours. The duration of the effect of HS-20001, HS-20002, HS-20003, HS-20004, HS-20005, HS-20006, HS-20007 and HS-20008 on decreasing random blood glucose in the dose of 10 μg/kg was more than 8 hours.

1-22. (canceled)

23. A GLP-1 analogue comprising an amino acid sequence of formula (I) or a pharmaceutically acceptable salt thereof:
X1-X2-Glu-Gly-Thr-Phe-Thr-Ser-Asp-X10-Ser-X12-X13-X14-Glu-X16-X17-Ala-X19-X20-X21-Phe-Ile-X24-Trp-Leu-X27-X28-X29-X30-X31-X32-X33-X34-X35-X36-X37-X38-X39-Lys Formula (I)
wherein the GLP-1 analogue includes a lipophilic substituent of formula R₁(CH₂)ₙ—CO— that is linked to the amino acid sequence of formula (I) through an amide bond;
where R₁ is selected from CH₃— and HOOC—;
n is an integer selected from 8-25;
each of X₁, X₂, X₁₀, X₁₂, X₁₃, X₁₄, X₁₆, X₁₇, X₁₉, X₂₀, X₂₁, X₂₄, X₂₇, X₂₈, X₂₉, X₃₀, X₃₁, X₃₂, X₃₃, X₃₄, X₃₅, X₃₆, X₃₇, X₃₈, and X₃₉ is independently selected from any natural or non natural amino acids or peptide segments consisting of any natural or non natural amino acids.

24. The GLP-1 analogue or a pharmaceutically acceptable salt thereof of claim 23, wherein X₁ is selected from L-His and D-His;
X₂ is selected from Ala, D-Ala, Gly, Val, Leu, Ile, Lys and Aib;
X₁₀ is selected from Val and Leu;
X₁₂ is selected from Ser, Lys and Arg;
X₁₃ is selected from Tyr and Gln;
X₁₄ is selected from Leu, Met, and Nle;
X₁₆ is selected from Gly, Glu and Aib;
X₁₇ is selected from Gln, Glu, Lys and Arg;
X₁₉ is selected from Ala and Val;
X₂₀ is selected from Lys, Glu and Arg;
X₂₁ is selected from Glu and Leu;
X₂₄ is selected from Val, Lys, Glu, and Ala;
X₂₈ is selected from Lys, Glu, Asn and Arg;
X₂₉ is selected from Gly and Aibl;
X₃₀ is selected from Arg, Gly and Lys;
X₃₁ is selected from Gly, Ala, Glu, Pro and Lys;
X₃₂ is selected from Lys and Ser;
X₃₃ is selected from Lys and Ser;
X₃₄ is selected from Gly, Ala and Sar;
X₃₅ is selected from Gly, Ala and Sar;
X₃₆ is selected from Pro and Gly;
X₃₇ is selected from Pro and Gly;
X₃₈ is selected from Pro and Gly;
X₃₉ is selected from Ser and Tyr.

25. The GLP-1 analogue or a pharmaceutically acceptable salt thereof of claim 24, wherein the amide bond is formed by the lipophilic substituent of formula R₁(CH₂)ₙ—CO— and an c amino group of the C-terminal Lys residue.

26. The GLP-1 analogue or a pharmaceutically acceptable salt thereof of claim 25, wherein R₁ is CH₃—.

27. The GLP-1 analogue or a pharmaceutically acceptable salt thereof of claim 26, wherein n is selected from 8, 10, 12, 14, 16, 18, 20, and 22.

28. The GLP-1 analogue or a pharmaceutically acceptable salt thereof of claim 26, wherein n is 14.

29. The GLP-1 analogue or a pharmaceutically acceptable salt thereof of claim 25, wherein R₁ is HOOC—.

30. The GLP-1 analogue or a pharmaceutically acceptable salt thereof of claim 29, wherein n is selected from 8, 10, 12, 14, 16, 18, 20, and 22.

31. The GLP-1 analogue or a pharmaceutically acceptable salt thereof of claim 29, wherein n is 14.

32. The GLP-1 analogue or a pharmaceutically acceptable salt thereof of claim 24, wherein the amide bond is formed by the lipophilic substituent of formula R₁(CH₂)ₙ—CO— and an α amino group of the C-terminal Lys residue.

33. The GLP-1 analogue or a pharmaceutically acceptable salt thereof of claim 32, wherein R₁ is CH₃—.

34. The GLP-1 analogue or a pharmaceutically acceptable salt thereof of claim 33, wherein n is selected from 8, 10, 12, 14, 16, 18, 20, and 22.

35. The GLP-1 analogue or a pharmaceutically acceptable salt thereof of claim 33, wherein n is 14.

36. The GLP-1 analogue or a pharmaceutically acceptable salt thereof of claim 32, wherein R₁ is HOOC—.

37. The GLP-1 analogue or a pharmaceutically acceptable salt thereof of claim 36, wherein n is selected from 8, 10, 12, 14, 16, 18, 20, and 22.

38. The GLP-1 analogue or a pharmaceutically acceptable salt thereof of claim 36, wherein n is 14.

39. The GLP-1 analogue or a pharmaceutically acceptable salt thereof of claim 23, wherein the amino acid sequence of Formula (I) is selected from the group consisting of: SEQ ID NO: 1 to SEQ ID NO: 120.

40. The GLP-1 analogue or a pharmaceutically acceptable salt thereof of claim 23, wherein the amino acid sequence of Formula (I) is selected from the group consisting of SEQ ID NO: 1 to SEQ ID NO: 20.

41. The GLP-1 analogue or a pharmaceutically acceptable salt thereof of claim 23, wherein the amino acid sequence of Formula (I) is selected from the group consisting of SEQ ID NO: 1 to SEQ ID NO: 8.

42. The GLP-1 analogue or a pharmaceutically acceptable salt thereof of claim 23, wherein the amino acid sequence of Formula (I) is SEQ ID NO: 4.

43. A pharmaceutical composition comprising:
(1) a therapeutically effective amount of a GLP-1 analogue or a pharmaceutically acceptable salt thereof comprising an amino acid sequence of formula (I):
X₁-X₂-Glu-Gly-Thr-Phe-Thr-Ser-Asp-X₁₀-Ser-X₁₂-X₁₃-X₁₄-Glu-X₁₆-X₁₇-Ala-X₁₉-X₂₀-X₂₁-Phe-Ile-X₂₄-Trp-Leu-X₂₇-X₂₈-X₂₉-X₃₀-X₃₁-X₃₂-X₃₃-X₃₄-X₃₅-X₃₈-X₃₇-X₃₈-X₃₉-Lys Formula (I)
wherein the GLP-1 analogue includes a lipophilic substituent of formula R₁(CH₂)ₙ—CO— that is linked to the amino acid sequence of formula (I) through an amide bond;
where R₁ is selected from CH₃— and HOOC—;
n is an integer selected from 8-25;
each of X₁, X₂, X₁₀, X₁₂, X₁₃, X₁₄, X₁₆, X₁₇, X₁₉, X₂₀, X₂₁, X₂₄, X₂₇, X₂₈, X₂₉, X₃₀, X₃₁, X₃₂, X₃₃, X₃₄, X₃₅, X₃₆, X₃₇, X₃₈, and X₃₉ is independently selected from any natural or non natural amino acids or peptide segments consisting of any natural or non natural amino acids; and
(2) a pharmaceutically acceptable excipient or drug carrier.

44. A method of treating non-insulin-dependent diabetes mellitus, insulin-dependent diabetes, or obesity comprising administering a therapeutically effective amount of a GLP-1 analogue or a pharmaceutically acceptable salt thereof comprising an amino acid sequence of formula (I):
X₁-X₂-Glu-Gly-Thr-Phe-Thr-Ser-Asp-X₁₀-Ser-X₁₂-X₁₃-X₁₄-Glu-X₁₆-X₁₇-Ala-X₁₉-X₂₀-X₂₁-Phe-Ile-X₂₄-Trp-Leu-X₂₇-X₂₈-X₂₉-X₃₀-X₃₁-X₃₂-X₃₃-X₃₄-X₃₅-X₃₆-X₃₇-X₃₈-X₃₉-Lys Formula (I)
wherein the GLP-1 analogue includes a lipophilic substituent of formula R₁(CH₂)ₙ—CO— that is linked to the amino acid sequence of formula (I) through an amide bond;
where R₁ is selected from CH₃— and HOOC—;
n is an integer selected from 8-25;
each of X₁, X₂, X₁₀, X₁₂, X₁₃, X₁₄, X₁₆, X₁₇, X₁₉, X₂₀, X₂₁, X₂₄, X₂₇, X₂₈, X₂₉, X₃₀, X₃₁, X₃₂, X₃₃, X₃₄, X₃₅, X₃₆, X₃₇, X₃₈, and X₃₉ is independently selected from any natural or non natural amino acids or peptide segments consisting of any natural or non natural amino acids.
